(19) United States
(12) Patent Application Publication
(10) Pub. No.: US 2014/0011812 A1
(43) Pub. Date: Jan. 9, 2014

(54) Methods of Treating Inflammation

(71) Applicants: Aviv Regev, Cambridge, MA (US); Ido Amit, Rehovot (IL); Nir Hacohen, Brookline, MA (US); Manuel Garber, Winchester, MA (US); Nicolas Chevrier, Boston, MA (US)

(72) Inventors: Aviv Regev, Cambridge, MA (US); Ido Amit, Rehovot (IL); Nir Hacohen, Brookline, MA (US); Manuel Garber, Winchester, MA (US); Nicolas Chevrier, Boston, MA (US)

(73) Assignees: The Broad Institute, Inc., Cambridge, MA (US); Massachusetts Institute of Technology, Cambridge, MA (US); President and Fellows of Harvard College, Cambridge, MA (US); The General Hospital Corporation d/b/a Massachusetts General Hospital, Boston, MA (US)

(21) Appl. No.: 13/878,386

(22) PCT Filed: Oct. 7, 2011
(86) PCT No.: PCT/US11/55437

§ 371 Date: Sep. 25, 2013

Related U.S. Application Data
(60) Provisional application No. 61/391,490, filed on Oct. 8, 2010.
(60) Provisional application No. 61/497,251, filed on Jun. 15, 2011.

(51) Int. Cl. A61K 31/519; C12Q 1/68

(52) U.S. Cl. CPC A61K 31/519; C12Q 1/6883
USPC 514/249; 435/375; 435/6.13; 506/9; 435/5

(57) ABSTRACT

The present invention relates to methods of decreasing inflammation by inhibiting polo-like kinase (PlK)

### RELATED APPLICATIONS

This application claims the benefit of provisional applications U.S. Ser. No. 61/391,490, filed Oct. 8, 2010 and USSN, 61/497,251 filed Jun. 15, 2011, the contents which are each herein incorporated by reference in their entirety.

### STATEMENT REGARDING FEDERALLY SPONSORED RESEARCH

This invention was made with government support under AI057159 awarded by the National Institutes of Health. The government has certain rights in the invention.

### FIELD OF THE INVENTION

The present invention relates to the treating and/or preventing inflammation associated with an innate immune response to a pathogen.

### BACKGROUND OF THE INVENTION

Cells process environmental signals via signaling and transcriptional networks that culminate with appropriate regulation of output genes. Subtle changes in these networks underlie human diseases, making the elucidation of pathway components and architecture one of the major goals in the post-genome era. For example, innate immune dendritic cells (DCs) rely on multiple sensors, including Toll-like receptors (TLRs), to detect infectious and danger signals before mounting specific immune responses by instructing lymphocytes (Takeuchi & Akira, Pattern recognition receptors and inflammation, Cell, 2010). Defects at the level of input, signal processing, or output of these pathogen-sensing pathways are the underlying causes of many diseases due to their central role in regulating inflammatory processes (Medzhitov, Inflammation 2010: new adventures of an old flame, Cell, 2010). Filling the gaps in our knowledge of these pathways is a critical pre-requisite to future, successful manipulations of the immune system.

Upon activation, signaling networks such as the TLR system not only induce expression of effector genes (e.g., interferons against viral infections), but also induce genes whose products are required for signal propagation and extinction. One example of the latter form of inducible gene in the TLR system is Tnfaip3 (A20), which is known to terminate NF-κB-mediated signals and therefore limit inflammation (Lee et al., Failure to regulate TNF-induced NF-κB and cell death responses in A20-deficient mice, Science, 2000). Moreover, mutations in the human Tnfaip3 locus has been linked to multiple disorders ranging from cancer to lupus, or diabetes. These types of feedback from induced transcripts can also occur by direct optimization of cytoplasmic signaling components. Given this property of signaling networks to optimize the activity and expression of its very own components, we hypothesized that signaling regulators of a network can be extracted from its transcriptional output. Here we verify our hypothesis in the TLR system of DCs and validate a systematic strategy for the identification of signaling regulators. First, both known and candidate signaling regulators of the TLR network were extracted from genome-wide expression profiles from DCs stimulated with pathogen mimics. Second, the expression of TLR signature output genes was measured upon perturbation of selected signaling regulators (Amit et al., Unbiased reconstruction of a mammalian transcriptional network mediating pathogen responses, Science, 2009). Using this approach, we correctly assigned functions to six known TLR signaling components and highlight a level of cross talks between these components higher than previously thought. In addition, we identified and functionally validated seventeen new signaling regulators of the TLR network. Among these new regulators, Polo-like kinase (PLK) family member 2 and 4 are cell cycle regulators that are co-opted by anti-viral pathways of innate immune DCs. Lastly, chemical perturbations of PLKs demonstrate the potential of our approach in drug target discovery.

### SUMMARY OF THE INVENTION

The invention provides methods of decreasing inflammation associated with an innate immune response to a pathogen or pathogen derived molecule by administering to a subject in need thereof a polo-like kinase (Plk) inhibitor. The pathogen is a virus or a component thereof. In some aspects the pathogen binds to a toll-like receptor on the surface or in endomes of a dendritic cell or a cytosolic RIG-1 like receptor of a dentritic cell.

In another aspect the invention provides a method of treating inflammation by administering to a subject in need thereof a polo-like kinase (Plk) inhibitor. The inflammation is a symptom of a disease selected from the group consisting of viral infection, bacteria infection, autoimmune disease, or mucositis.

The invention further provides method of decreasing anti-viral cytokine expression by a dendritic cell by contacting the cell with a polo-like kinase (Plk) inhibitor. In yet another aspect the invention provides a method of decreasing anti-viral cytokine expression in a subject by administering to a subject in need thereof a polo-like kinase (Plk) inhibitor. The cytokine is interferon-β or CXCL-10.

The Plk inhibitor is specific for at least two Plks. For example, the Plk inhibitor is specific for at least Plk2 and Plk4. Alternatively, the Plk the inhibitor is a pan-specific Plk inhibitor. Preferably, the Plk inhibitor is BI 2536, poloxipan, or GW843682X.

In a further aspect the invention provides a method of indentifying genes or genetic elements associated with a pathogen specific response by contacting a dendritic cell with a toll-like receptor agonist; and identifying a gene or genetic element whose expression is modulated by the toll-like receptor agonist. Optionally the method further comprises perturbing expression of the gene or genetic element identified and determining a gene whose expression is modulated the perturbation. The toll-like receptor agonist is Pam3CSK4, lipopolysaccharide, polyinosinic: polycytidylic acid, gardiquimod, or CpG. The pathogen is a virus, a bacterium, a fungus or a parasite.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1. mRNAs of Signaling Components are Differentially Regulated Upon Toll-Like Receptor (TLR) Stimulation

(A) Simplified schematic of the TLR2, 3, and 4 pathways (Takeuchi and Akira, 2010). (B) mRNA expression profiles of differentially expressed signaling genes. Shown are expression profiles for 280 differentially expressed signaling genes (rows) at different time points (columns): a control time course (no stimulation, Ctrl) and following stimulations with Pam3CSK4 (PAM), lipopolysaccharide (LPS), and poly(I:C). Tick marks: time point post-stimulation (0.5, 1, 2, 4, 6, 8, 12, 16, 24 h). Shown are genes with at least a 1.7 fold change in expression compared to pre-stimulation levels in both duplicates of at least one time point. The three leftmost columns indicate kinase (KIN), phosphatase (PSP), and signaling regulators (SIG) (black bars). Values from duplicate arrays were collapsed and gene expression profiles were hierarchically clustered. The rightmost color-coded column indicates the 5 major expression clusters. (C and D) mRNA expression profiles of candidate (C) and canonical (D) TLR signaling regulators selected for subsequent experiments. The color-coding of the gene names highlight the corresponding expression cluster from the complete matrix from A.

FIG. 2. A Perturbation Strategy Assigns Function to Signaling Components within the TLR Pathways

(A) Perturbation profiles of six canonical (purple) and 17 candidate (blue) signaling components, and 20 core TLR transcriptional regulators belonging to the inflammatory (orange) and the antiviral (green) programs. Shown are the perturbed regulators (columns) and their statistically significant effects (False discovery rate, FDR<0.02) on each of the 118 TLR signature genes (rows). Red: significant activating relation (target gene expression decreased following perturbation); blue: significant repressing relation (target gene expression increased following perturbation); white: no significant effect. The right-most column categorizes signature genes into antiviral (light grey) and inflammatory (dark grey) programs. (B) Functional characterization based on similarity of perturbation profiles. Shown is a correlation matrix of the perturbation profiles from A. Yellow: positive correlation; purple: negative correlation; black: no correlation.

FIG. 3. Crkl Adaptor Functions in the Antiviral Arm of TLR4 Signaling

(A) Comparison of Crkl and Mapk9 knockdown profiles. Shown are the effects of Crkl and Mapk9 perturbation (columns) on the 118 signature genes (rows). Data was extracted from FIG. 2A. (B) Inhibition of transcription of antiviral cytokines in Crkl^{-/-} BMDCs. Shown are mRNA levels (qPCR; relative to t=0) for Ifnb1 (left), Cxcl10 (middle) and Cxcl1 (right) in three replicates per time point. Error bars represent the SEM (n=3 mice). (C) Crkl phosphorylation is induced following LPS stimulation. Top: Schematic depiction of experimental workflow. From left: Protein lysates from unstimulated (Control) and LPS-treated BMDCs grown in “light” and “heavy” SILAC medium were mixed (1:1) and digested into peptides with trypsin before phospho-tyrosine (pY) peptide enrichment by immunoprecipitation, and LC-MS/MS analysis. Bottom: Shown are the differential phosphorylation levels (log 2 ratios, Y axis) of all 62 phosphopeptides identified and quantified by LC-MS/MS (X axis). Black: peptides with more than 2 fold differential expression (left: induced; right: repressed).

FIG. 4. Polo-Like Kinase (Plk) 2 and 4 Regulate the Antiviral Program

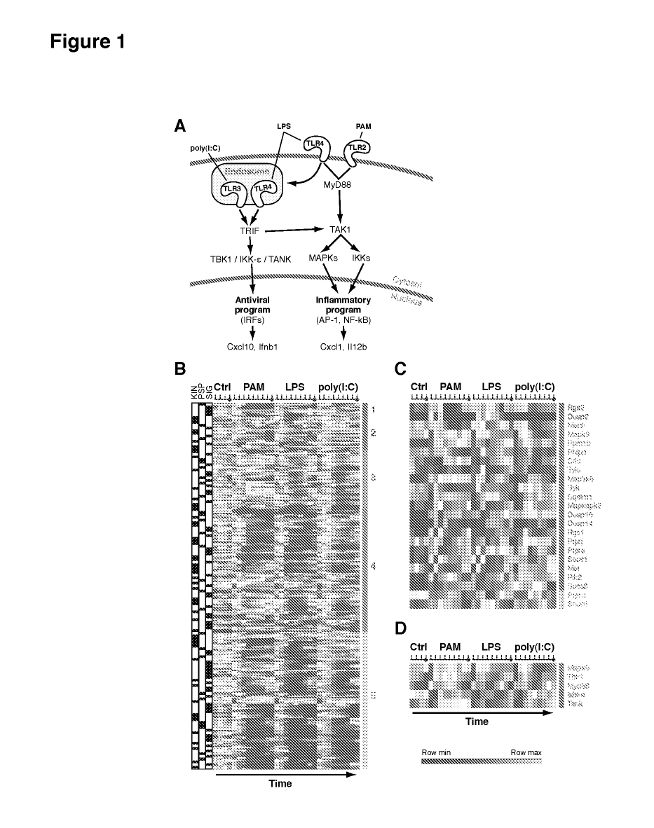

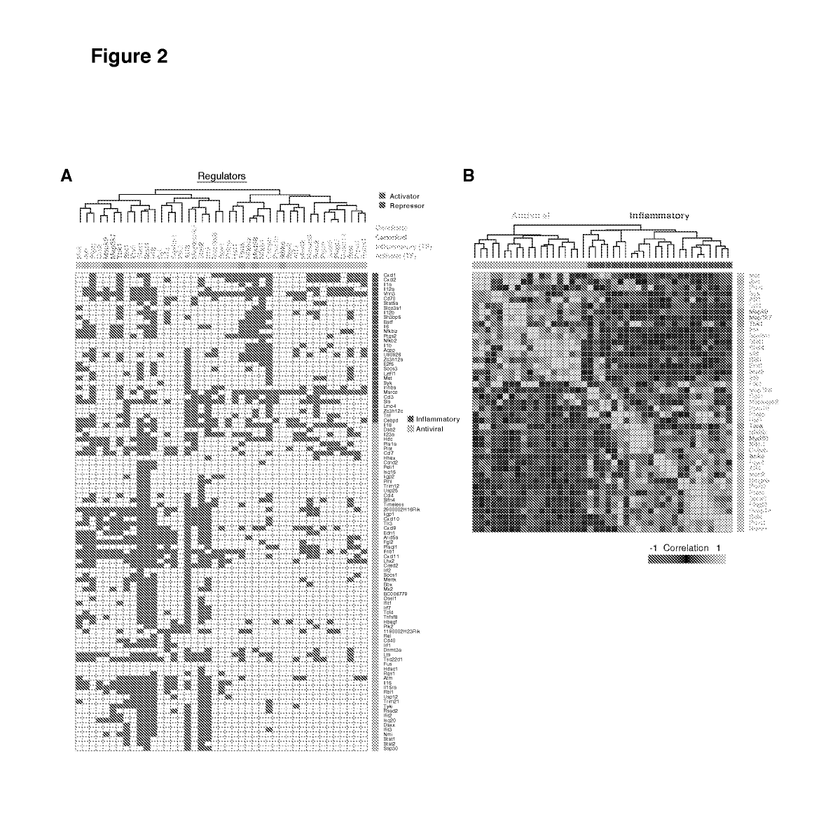

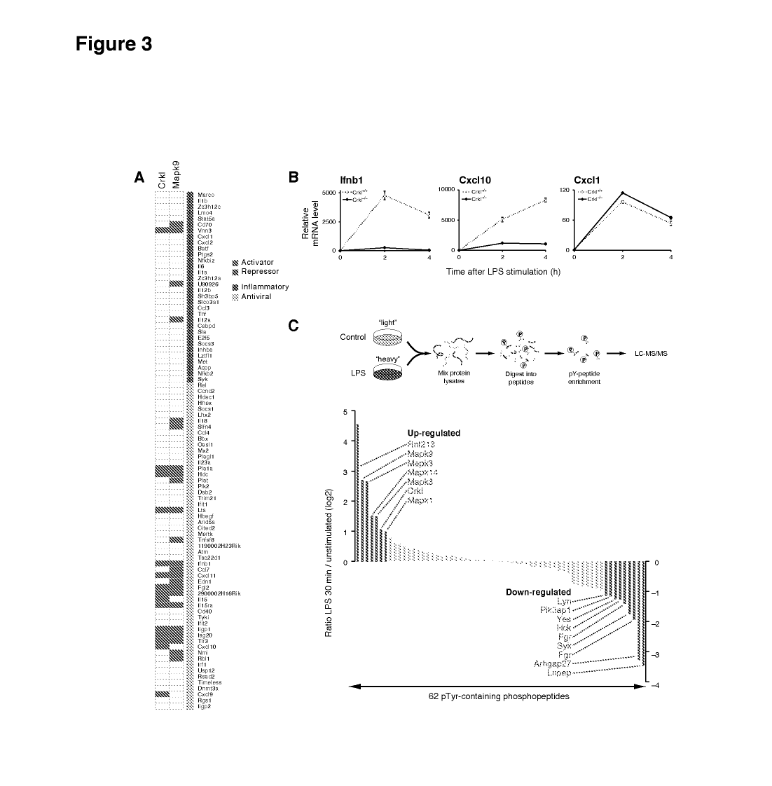

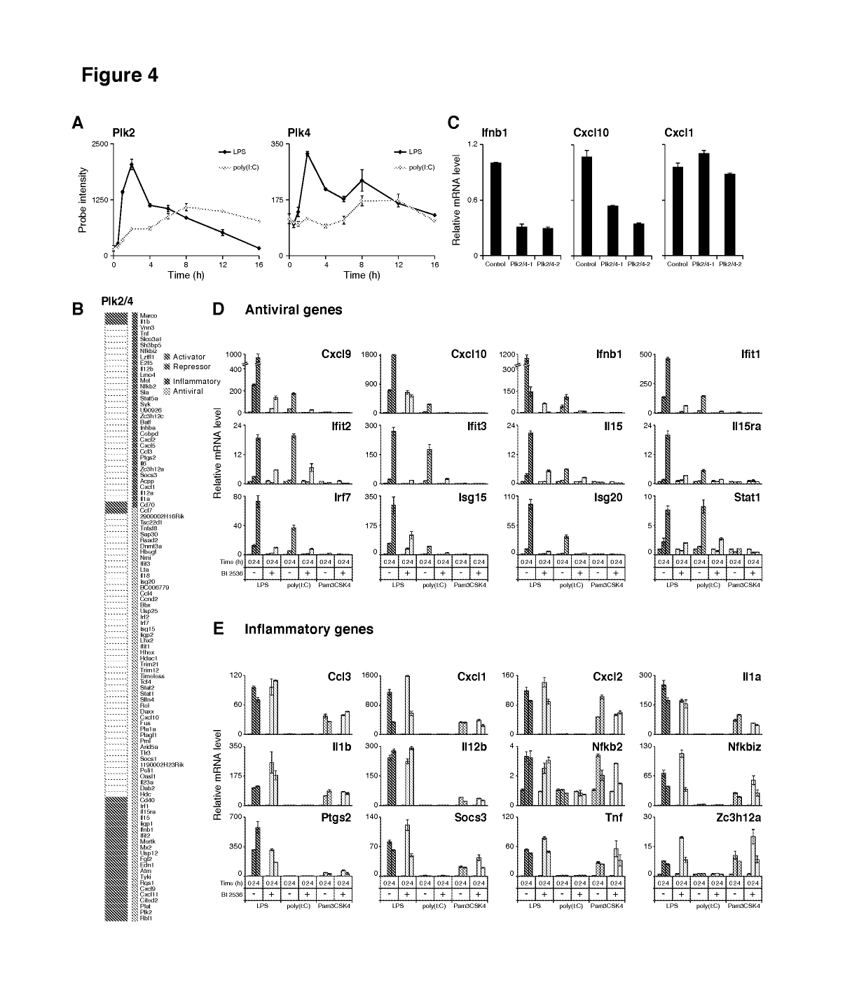

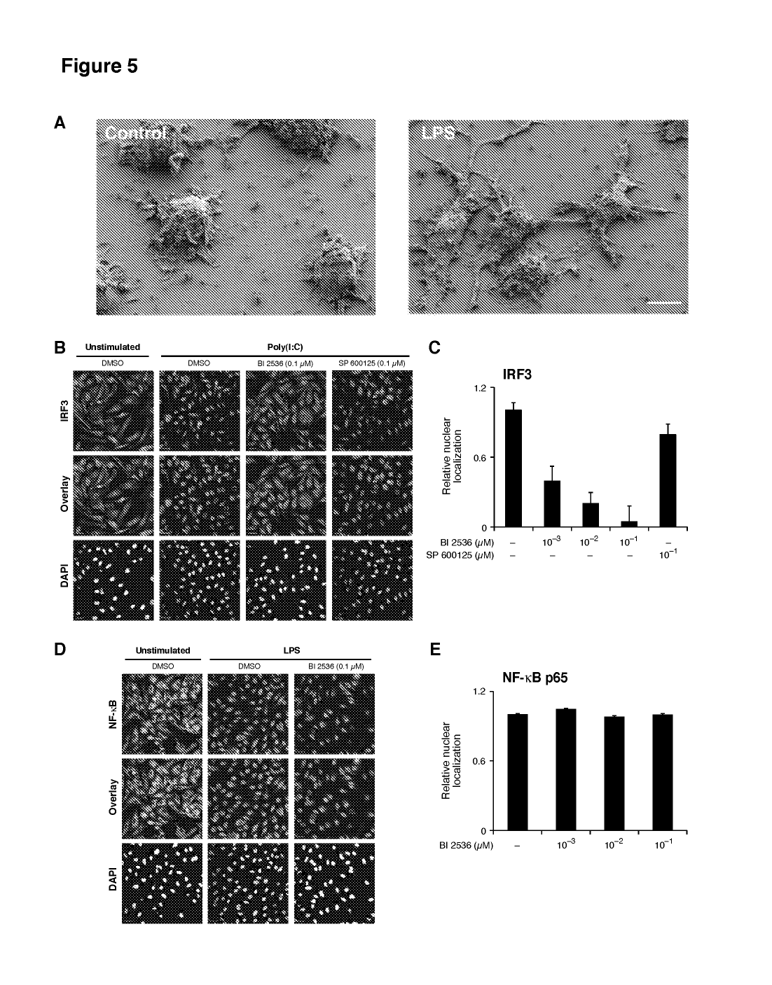

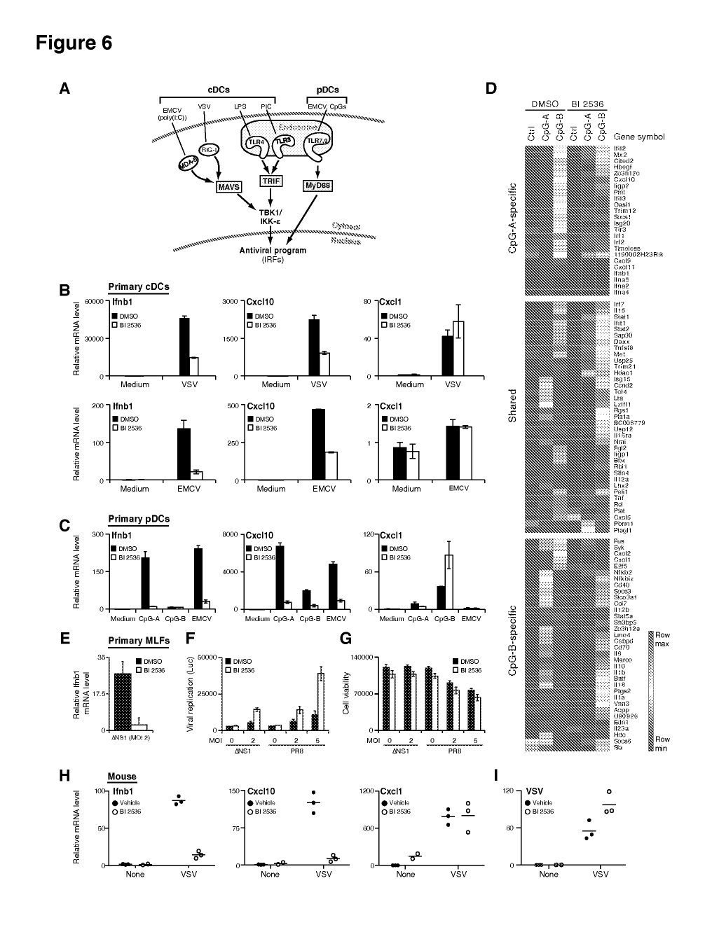

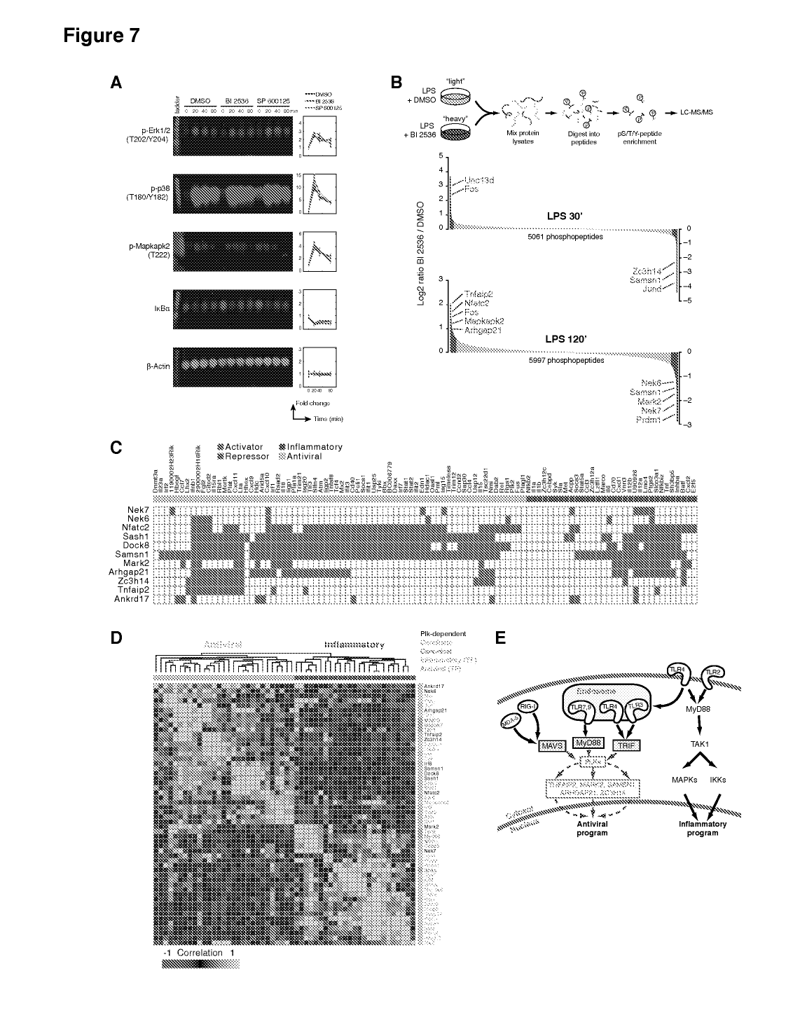

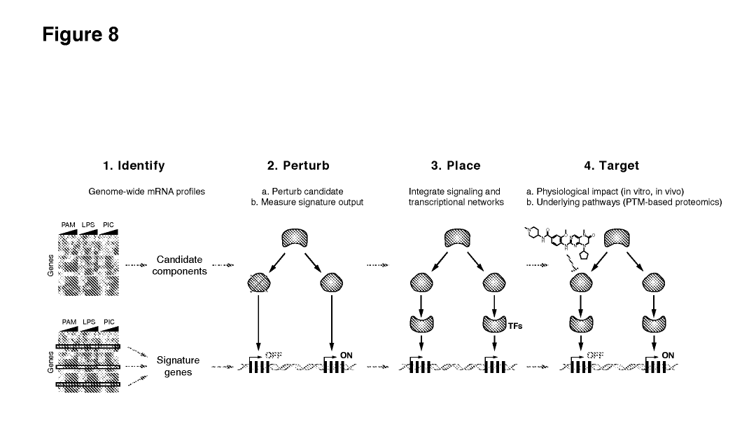

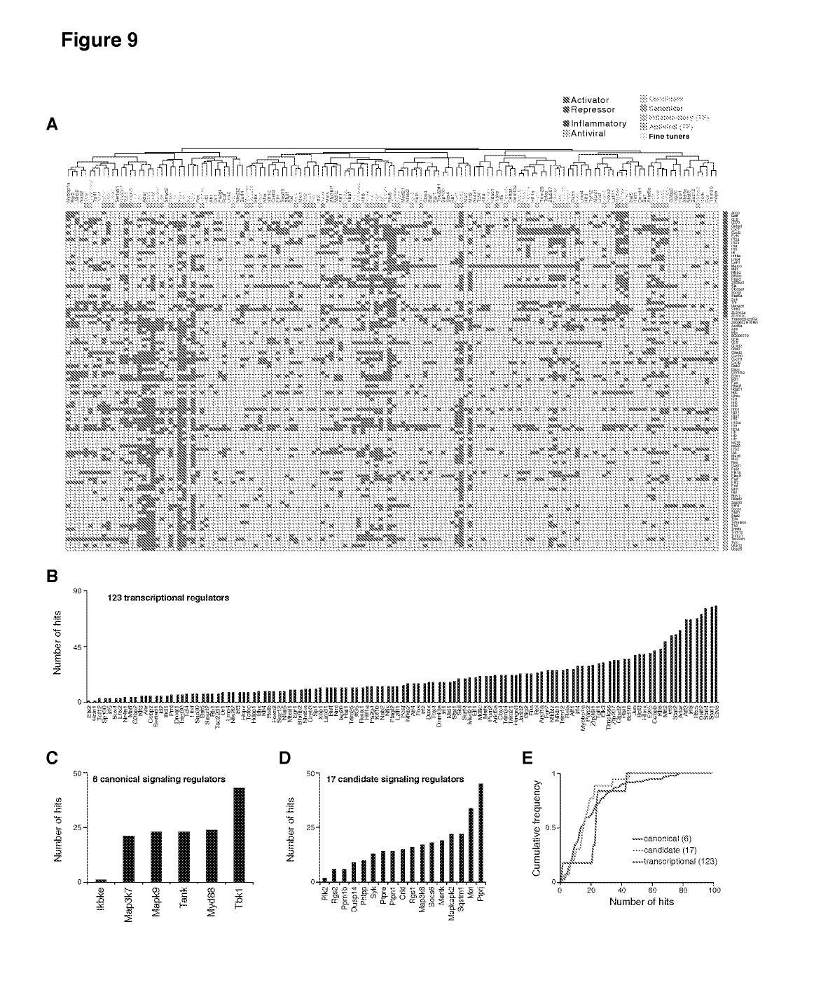

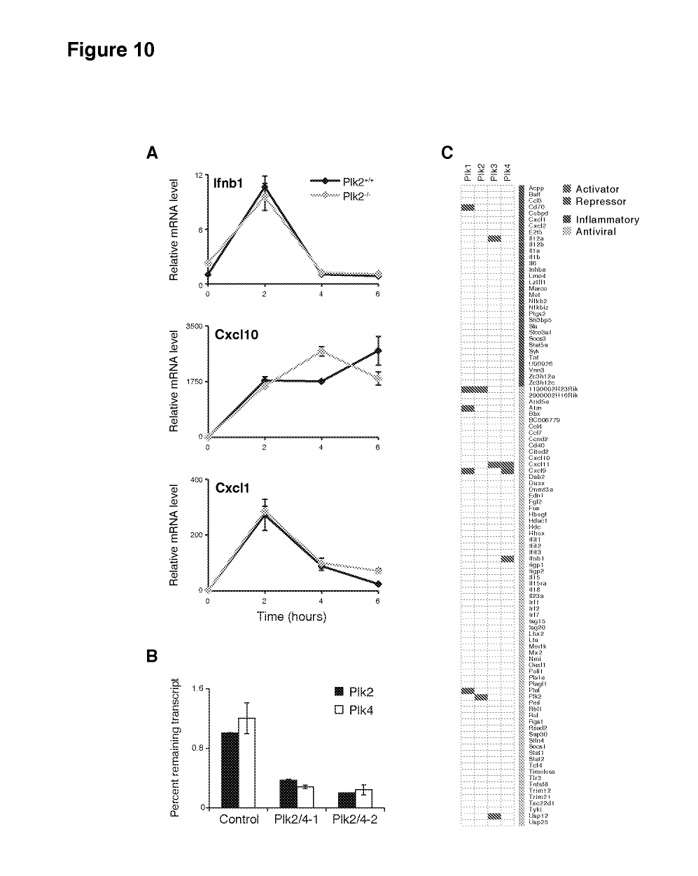

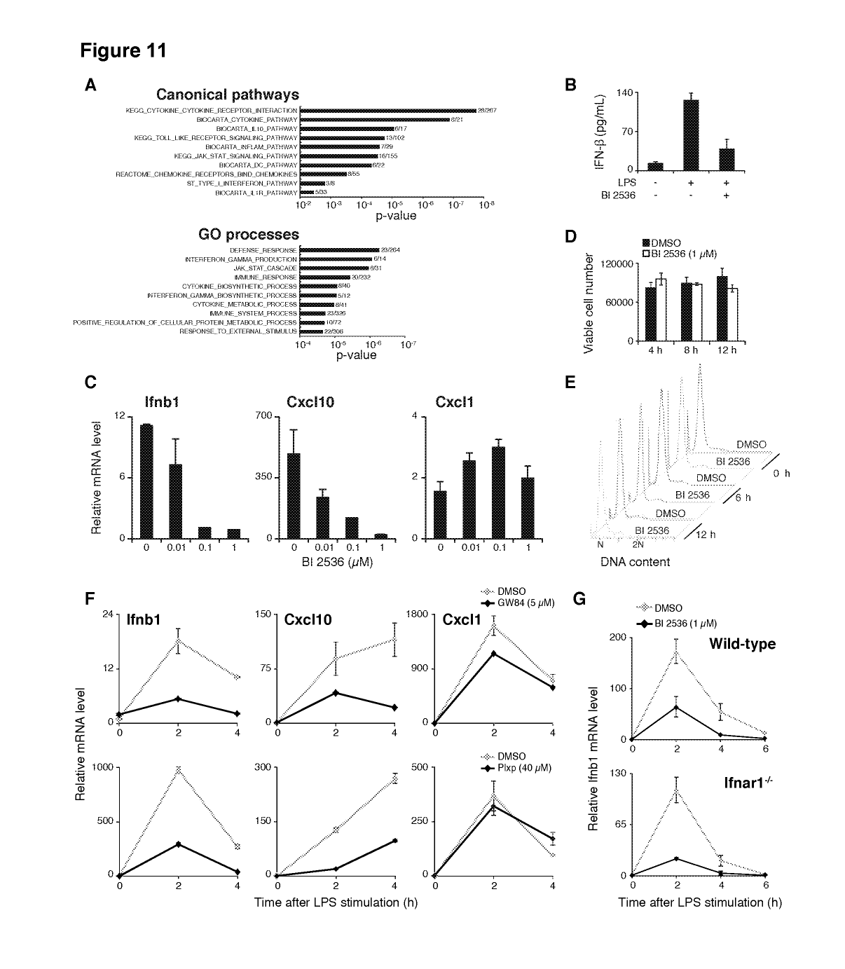

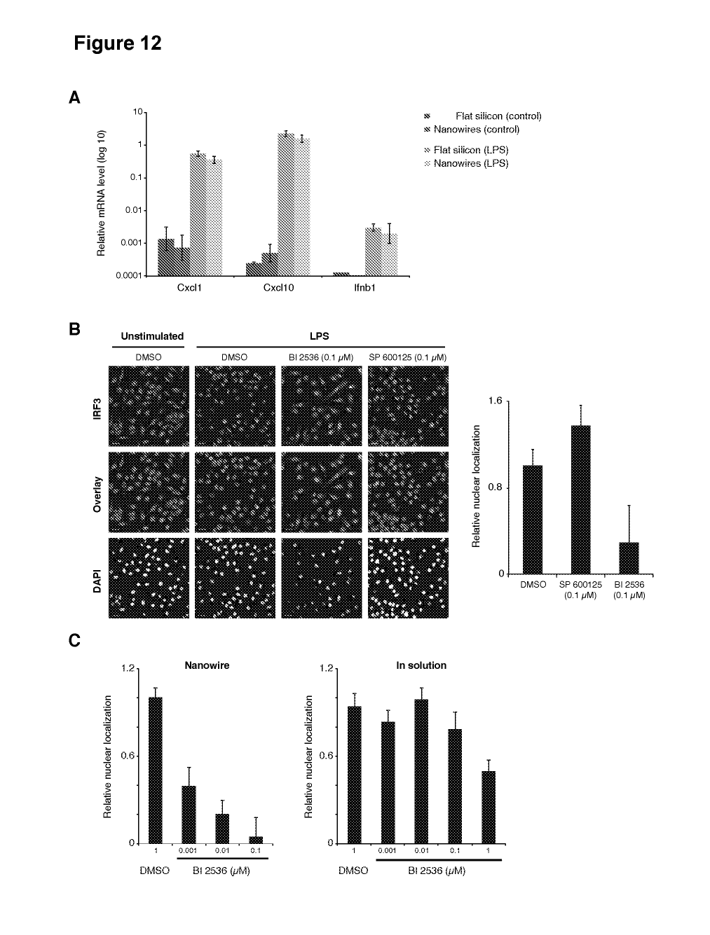

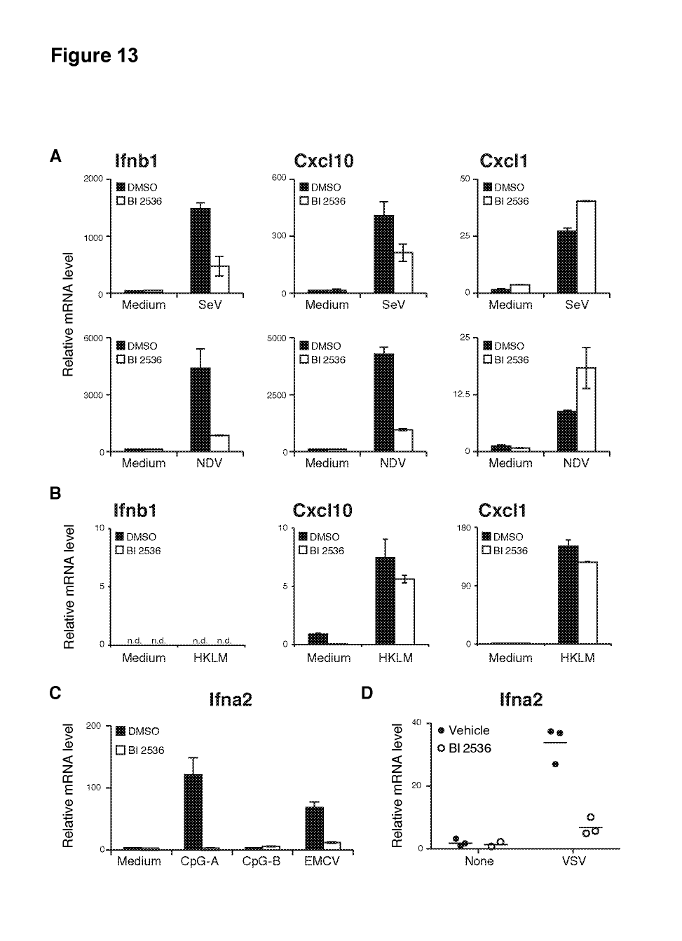

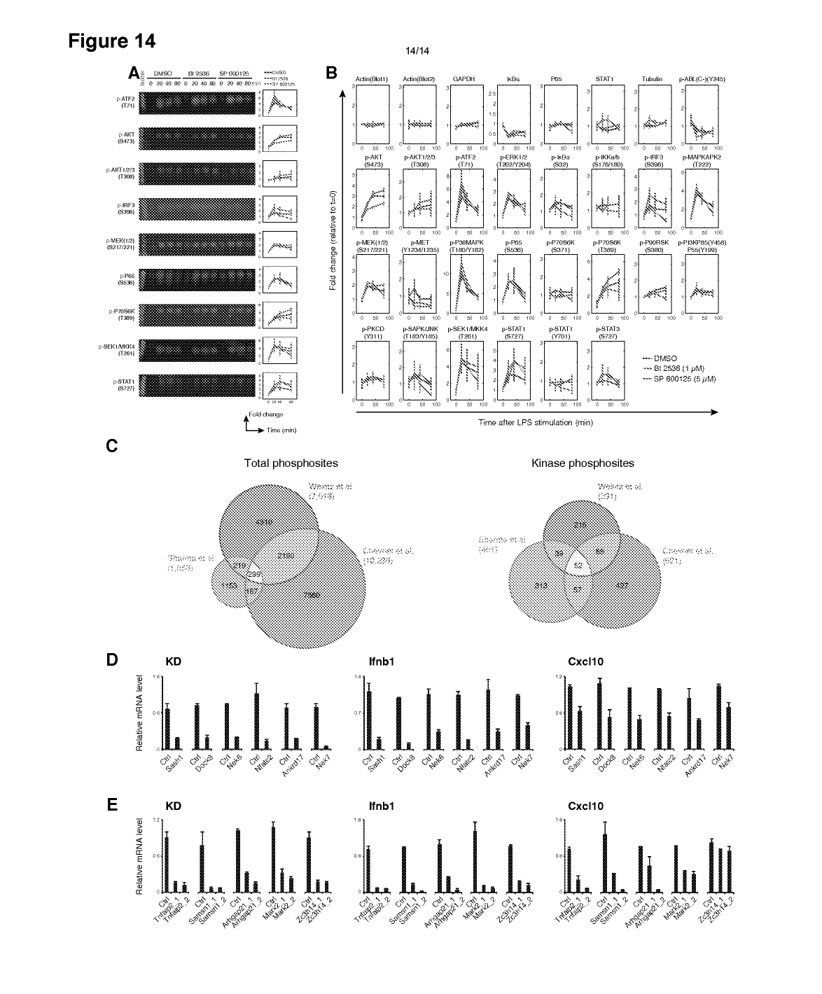

(A) Similarity of Plk2 and Plk4 mRNA expression profiles. Shown are mRNA levels (from FIG. 1B) of Plk2 (left) and Plk4 (right) following stimulation with LPS (black) or poly(I:C) (grey). (B) Double knockdown of Plk2 and 4 represses the antiviral signature. Shown are significant changes in expression of TLR signature genes (rows) following double knockdown of Plk2 and 4. Red and blue mark significant hits as in FIG. 2, only for genes where the effect was consistent between the two independent combinations of shRNAs. (C) Double knockdown of Plk2 and 4 represses antiviral cytokine mRNAs. Shown are expression levels (qPCR) relative to control shRNAs (Control) for two antiviral cytokines (Ifnb1 and Cxcl10) and for an inflammatory cytokine (Cxcl1), following LPS stimulation in BMDCs using two independent combinations of shRNAs (Plk2/4-1, Plk2/4-2). Three replicates for each experiment; error bars are the SEM. (D and E) BI 2536 specifically abrogates transcription of antiviral genes without affecting inflammatory genes following stimulation with LPS, poly(I:C), or Pam3CSK4. Shown are mRNA levels (qPCR; relative to t=0) for 12 indicated antiviral (D) and 12 inflammatory (E) genes in BMDCs treated with BI 2536 (1 μM; dark color bars) or DMSO vehicle (light color bars) and stimulated for 0, 2 or 4 h with LPS (dark and light

FIG. 5. BI 2536-Mediated Plk Inhibition Blocks IRF3 Nuclear Translocation in DCs

(A) DCs on nanowires (NW) undergo normal morphological changes upon LPS stimulation. Shown are electron micrographs of BMDCs plated on bare vertical silicon NW that were left unstimulated (left; Control) or stimulated with LPS (right). Scale bars, 5 μm. (B-E) BI 2536 inhibits IRF3, but not NF-κB p65, nuclear translocation following TLR stimulation. (B and D) Shown are confocal micrographs of BMDCs plated on vertical silicon NW pre-coated with vehicle control (DMSO; B and D), Plk inhibitor (BI 2536; B and D), or control Jnk inhibitor (SP 600125; B), and stimulated with poly(I:C) for 2 h (B) or LPS for 30 min (D) (reflecting peak time of nuclear translocation for IRF3 and NF-κB p65, respectively), or left unstimulated (B and D). Cells were analyzed for DAPI (B and D), IRF3 (B) and NF-κB p65 subunit (D) staining. Scale bars, 5 μM. (C and E) Nuclear translocation (from confocal micrographs) of IRF3 (C) and NF-κB p65 (E) was quantified using DAPI staining as a nuclear mask (purple circles; overlay in B and D) to determine the ratio of total versus nuclear fluorescence (Y axis) in BMDCs cultured on NW coated with different amounts of BI 2536 or SP 600125, or with vehicle control (DMSO; X axis). Three replicates in each experiment; error bars are the SEM.

FIG. 6. Plks are Critical in the Induction of Type I Interferons In Vitro and In Vivo.

(A) IFN-inducing pathways in conventional DCs (cDCs) and plasmacytoid DCs (pDCs). (B, C) BI 2536 inhibits mRNA levels for antiviral cytokines in response to diverse stimuli in cDCs and pDCs. Shown are Ifnb1, Cxcl10 and Cxcl1 mRNA levels (qPCR; relative to t=0) in cells treated with BI 2536 (1 μM; white bars) or DMSO vehicle (black bars) in cDCs (B) infected with VSV (MOI 1; B top) or with EMCV (MOI 10; B bottom), and in pDCs (C) stimulated with CpG type A or B, or infected with EMCV (MOI 10). Three replicates in each experiment; error bars are the SEM. (D) BI 2536 inhibits the CpG-A response, but has little effect on the CpG-B response. Shown are mRNA levels (nCounter) for the 118 TLR signature genes (rows) in pDCs treated with DMSO vehicle or BI 2536 (1 μM) and left untreated (Ctrl) or stimulated with CpG-A or -B for 6 h (columns). Three clusters of genes are shown: CpG-A-specific (top), CpG-B-specific (bottom), and shared by CpG-A and -B (middle). (E-G) BI 2536 inhibits IFN-β production in primary mouse lung fibroblasts (MLFs), leading to an increase in viral replication. MLFs treated with BI 2536 (1 μM; white bars) or vehicle control (DMSO; black bars) were infected with influenza ΔNS1 or PR8 strains at indicated MOIs. Shown are Ifnb1 mRNA levels measured by qPCR (relative to t=0; E), viral replication as measured by luciferase (Luc) activity in reporter cells (F), and cell viability measured by CellTiter-Glo assay (G). (H and I) BI 2536 inhibits antiviral cytokine mRNA production, while increasing viral replication during in vivo VSV infection. Shown are Ifnb1, Cxcl10 and Cxcl1 mRNA (H), and VSV viral RNA (I) levels (qPCR; relative to uninfected animals) from popliteal lymph nodes of mice injected with BI 2536 (white circles) or DMSO vehicle (black circles) prior to and during the course of infection with VSV (intra-footpad). Nodes were harvested six hours post-infection. Each circle represents one animal (n=3). Data is representative of three independent experiments for each condition.

FIG. 7. Unbiased Phosphoproteomics Identifies a Novel Plk-Dependent Antiviral Pathway.

(A) BI 2536 does not affect phosphorylation and protein levels of known TLR signaling nodes. Shown are representative MicroWestern Array (MWA; see Experimental Procedures) blots (left) obtained from analyzing lysates from BMDCs pre-treated with DMSO, BI 2536 (1 or SP 600125 (5 μM) and stimulated with LPS for 0, 20, 40, 80 min. Blots were analyzed using indicated antibodies (left most), and fold change in fluorescence signals was quantified relative to t=0 (right). Error bars are the SEM of triplicate MWA blots. (B) BI 2536 affects protein phosphorylation levels during LPS stimulation. Top: Schematic depiction of experimental workflow. From left to right: LPS-stimulated BMDCs cultured in “heavy” or “light” SILAC medium were pre-treated with BI 2536 (1 μM) or DMSO, respectively. Protein lysates were mixed (1:1) and digested into peptides with trypsin, before phospho-serine, -threonine and -tyrosine (pS/T/Y) peptide enrichment, and LC-MS/MS analysis. Bottom: Shown are the differential phosphorylation levels (average log₂ ratios of two independent experiments; Y axis) of all 5061 and 5997 phosphopeptides respectively identified and quantified by LC-MS/MS (X axis) at 30 min (top) and 120 min (bottom) post-LPS stimulation. Dark grey: phosphopeptides with a significant change (P_{unadjusted}<0.001 for both time points; FDR₃₀ₘᵢₙ=0.05; FDR₁₂₀ₘᵢₙ=0.03; left: induced; right: repressed). Average ratios from phosphopeptides identified and quantified in two independent experiments are depicted. (C) Eleven Plk-dependent phosphoproteins significantly affect the expression of TLR signature genes. Shown are significant changes in expression of the TLR signature genes (rows) following knockdown of each of the 11 phosphoproteins (columns), following stimulation with LPS for 6 h. Red and blue mark significant hits (as presented in FIG. 2) and are shown only for genes where the effect was consistent between two independent experiments. (D) Functional characterization based on similarity of perturbation profiles. Shown is a correlation matrix of the perturbation profiles from C (grey), and those from FIG. 2B including canonical (purple) and candidate (blue) signaling components as well as core antiviral (green) and inflammatory (orange) transcriptional regulators. Yellow: positive correlation; purple: negative correlation; black: no correlation. (E) A Plk-dependent pathway in antiviral sensing. Shown is a diagram of a model of the Plk-dependent pathway of IFN induction in innate immunity. Out of the 11 Plk-dependent proteins described in C and D, only the 5 showing a phenotype with 2 independent shRNAs are depicted.

FIG. 8. A Systematic Approach to Dissect Signaling Pathways

Shown is a schematic depicting the strategy consisting of 4 steps (from left to right): (1) extract both candidate signaling regulators and signature genes; (2) perturb each candidate with shRNAs and measure the effect on the expression of signature genes; (3) compare perturbation profiles of signaling and transcriptional regulators to start assembling pathways; (4) use small molecule targeting of signaling nodes of interest to a) evaluate the physiological relevance of new signaling node, and b) identify underlying pathways by discovering downstream effector molecules.

FIG. 9. Perturbations of Signaling and Transcriptional Regulators have Similar Effects on the TLR Signature Genes

(A) Perturbation profiles of 6 canonical (purple) and 17 candidate (light blue) signaling regulators, and 123 transcriptional regulators (TF) partitioned into regulators of the inflammatory (orange) and antiviral (green) programs, and fine tuners (grey), as previously defined in Amit et al., 2009. Shown are the perturbed regulators (columns) and their statistically significant effects (False discovery rate, FDR<2%) on each of the 118 TLR signature genes (rows). Red: significant activating relation (target gene expression decreased following perturbation); blue: significant repressing relation (target gene expression increased following perturbation); white: no significant effect. The column on the right indicates whether signature genes belong to the antiviral (light grey) or the inflammatory (dark grey) programs.

(B-D) Shown are the numbers of signature genes hits (Y axis, ‘hits’) significantly affected by knockdown of each regulator (X axis) for the regulator categories shown in A: 123 transcriptional (B) and 6 previously known (C) and 17 candidate (D) signaling regulators.

(E) Candidate signaling regulators affect a similar number of ‘signature’ genes compared to transcriptional regulators. Shown is the cumulative distribution of the number of hits for the regulators shown in B-D.

FIG. 10. Individual Perturbation of Plk Family Members does not Affect TLR Output Gene Expression in DCs

(A) Plk2-deficient BMDCs respond to LPS similarly to wild-type cells. Shown are mRNA levels (qPCR; relative to t=0) for Ifnb1 (left), Cxcl10 (middle) and Cxcl1 (right) in three replicates per time point. Error bars represent the standard error of the mean. (B) Combinatorial knockdown levels of Plk2 and 4 in BMDCs. Shown are mRNA levels (qPCR), relative to non-targeting shRNAs (Control), of Plk2 and 4 in BMDCs using two independent combinations of shRNAs (Plk2/4-1 and -2). Three replicates in each experiment; error bars represent the standard error of the mean. (C) Perturbations of individual Plk family members do not affect TLR signature genes. Shown are the perturbed Plks (columns) and their statistically significant effects (FDR<2%) on each 118 TLR signature genes (rows). Red: significant activating relation (target gene expression decreased following perturbation); blue: significant repressing relation (target gene expression increased following perturbation); white: no significant effect. The column on the right indicates whether signature genes belong to the antiviral (light grey) or the inflammatory (dark grey) programs.

FIG. 11. BI 2536-Mediated Plk Inhibition Abrogates Antiviral Cytokine Production at the Protein and mRNA Levels, without Affecting the Viability and Cell Cycle Status of DCs

(A) Gene enrichment analysis of BI 2536-dependent genes from microarray measurements. Overlaps between the 311 unique genes downregulated 3-fold by BI 2536 treatment upon LPS or poly(I:C) stimulation, and Gene Ontology (GO) processes and canonical pathways (including the KEGG, REACTOME, and BIOCARTE databases present in the Molecular Signatures Database (MSigDB; see Experimental Procedures). Shown are P values (X axis) derived from the overlaps (n/N; top of each bar) between the number of queried genes (n) and genes present in indicated genesets (N). (B) BI 2536 strongly inhibits IFN-β secretion by BMDCs. Shown is IFN-β protein concentration (Y axis; measured by ELISA) in the supernatant of BMDCs treated with DMSO vehicle (−) or BI 2536 (1 μM; +), and stimulated with LPS (+) or left unstimulated (−) for 6 h. Three replicates in each experiment; error bars are the standard error of the mean. (C) BI 2536 inhibits antiviral cytokine mRNA production in a dose-dependent manner. Shown are mRNA levels (Y axis, qPCR; relative to vehicle control treatment) for two antiviral cytokines (Ifnb1, Cxcl10) and one inflammatory cytokine (Cxcl1) following LPS stimulation in BMDCs pre-treated with increasing amounts of BI 2536 (X axis). Three replicates in each experiment; error bars are the standard error of the mean. (D) BMDC viability is unaffected by Plk inhibition with BI 2536. Shown are viable cell numbers (Y axis, measured by Alamar blue; relative to a standard curve generated using a range of cell densities) after treatment with BI 2536 (white bars) or DMSO vehicle (black bars) at different time points following addition of BI 2536 (X axis). Three replicates in each experiment; error bars are the standard error of the mean. (E) The cell cycle state of BMDCs remains unchanged upon Plk inhibition with BI 2536. Shown are DNA contents (flow cytometry) of BMDCs stained with propidium iodide (PI) after treatment with BI 2536 or DMSO vehicle control for 0, 6, and 12 h. (F) Plk inhibitors structurally unrelated to BI 2536 also abrogate transcription of mRNAs for antiviral cytokines following stimulation with LPS. Shown are mRNA levels (qPCR; relative to t=0) for Ifnb1, Cxcl10 and Cxcl1 in BMDCs stimulated with LPS and treated with GW843682X (GW84; top) or Poloxipan (Plxp; bottom) (black line), or with DMSO vehicle (grey line) for 1 hour prior to stimulation. Three replicates for each experiment; error bars are the standard error of the mean. (G) Plks are directly downstream of TLR engagement. Shown are Ifnb1 mRNA levels (Y axis, qPCR; relative to t=j) following LPS stimulation for indicated times (X axis) in wild-type (top) and Ifnar1−/− (bottom) BMDCs treated with BI 2536 (1 μM; black) or vehicle control (DMSO; grey).

FIG. 12. BI 2536-Mediated Plk Inhibition Blocks IRF3 Nuclear Translocation in LPS-Stimulated DCs

(A) DCs plated on vertical silicon nanowires (NW) respond normally to TLR stimulation. Shown are cytokine mRNA levels (qPCR; relative to Gapdh mRNA) in BMDCs plated on NW or a flat silicon surface, and stimulated (LPS) or left untreated (control). Left to right: Cxcl1, Cxcl10, Ifnb1. Three replicates in each experiment; error bars are the standard error of the mean. (B) BI 2536 inhibits IRF3 nuclear translocation following LPS stimulation. Shown are confocal micrographs (left panel) of BMDCs plated on vertical silicon NW pre-coated with vehicle control (DMSO), Plk inhibitor (BI 2536), or control Jnk inhibitor (SP 600125), and stimulated with LPS for 45 min (reflecting peak time of nuclear translocation for IRF3 in the context of LPS stimulation), or left unstimulated. Cells were analyzed for DAPI and IRF3 staining. Scale bars, 5 μM. Nuclear translocation (from confocal micrographs) of IRF3 was quantified (right panel) using DAPI staining as a nuclear mask (purple circles on micrographs) to determine the ratio of total versus nuclear fluorescence (Y axis) in BMDCs cultured on NW coated with BI 2536, SP 600125, or vehicle control (DMSO; X axis). Three replicates in each experiment; error bars are the standard error of the mean. (C) Decrease in IRF3 nuclear translocation may be more efficient with NW-mediated delivery of BI 2536 than with delivery in solution. Shown are quantifications of confocal micrographs from BMDCs plated on vertical NW pre-coated with different amounts of BI 2536 (Nanowire; left panel) or left blank to allow in-solution delivery of BI 2536 (In solution; right panel). Cells were stimulated with poly(I:C) for 2 h prior to staining for DAPI and IRF3 as in B.

FIG. 13. Plks are Critical in Antiviral Responses In Vitro and In Vivo

(A) Plks are critical in RIG-1-mediated antiviral responses in vitro in DCs. Shown are mRNA levels (qPCR; relative to control, “medium”) in conventional DCs (GM-CSF-induced BMDCs) treated with BI 2536 (white bars) or DMSO vehicle (black bars), and infected at a multiplicity of infection (MOI) 1 with Sendai virus (SeV; top) or Newcastle disease virus (NDV; bottom). Three replicates in each experiments; error bars are the standard error of the mean. (B) Plk inhibition does not affect DC response to Listeria monocytogenes, a natural TLR2 agonist. Shown are mRNA levels (qPCR; relative to t=0) for Ifnb1, Cxcl10 and Cxcl1 in BMDCs stimulated with heat-killed Listeria monocytogenes (HKLM; MOI 5) and treated with BI 2536 (white bars), or with DMSO vehicle (black bars) for 1 hour prior to stimulation. Three replicates for each experiment; error bars are the standard error of the mean. (C) Plks are critical in type I interferon α2 (Ifna2) gene production by plasmacytoid DCs (pDCs). Shown is the mRNA level (qPCR; relative to control, “medium”) of Ifna2 in pDCs (Flt3L-induced BMDCs) treated with BI 2536 (1 μM; white bars) or DMSO control (black bars), and stimulated with CpG-A or -B, or infected with EMCV (MOI 10). Three replicates in each experiment; error bars are the standard error of the mean. (D) Plk inhibition in vivo inhibits type I IFN α2 production in the lymph node. Shown is Ifna2 mRNA level (qPCR; relative to uninfected animals) from popliteal lymph nodes of mice injected with BI 2536 (white circles) or DMSO vehicle (black circles) prior to and during the course of infection with VSV intra-footpad. Nodes were harvested six hours post-infection. Each circle represents one animal (n=3). Data is representative of two or three independent experiments for each condition.

FIG. 14. Plk Inhibition does not Affect Known TLR Signaling Components, but Affects 11 Newly Identified Plk-Dependent Phosphoproteins

(A, B) BI 2536-mediated Plk inhibition does not affect protein and/or phosphorylation levels of known TLR signaling nodes. (A) Shown are representative MicroWestern Array (MWA; see Experimental Procedures) blots obtained from analyzing lysates from BMDCs pre-treated with DMSO, BI 2536 (1 μM), or SP 600125 (5 μM) and stimulated with LPS for 0, 20, 40, 80 min. Blots were analyzed using indicated antibodies (left most), and fold change in fluorescence signals was quantified relative to t=0 (right; see Experimental Procedures). Error bars are the standard error of the mean of triplicate MWA blots. (B) Shown are the differential protein and phosphorylation levels (fold change; Y axis) of 6 proteins and 23 phosphosites in BMDCs treated with BI 2536 (red line), control JNK inhibitor (SP 600125; green line), or DMSO vehicle (blue line), and stimulated with LPS (0, 20, 40, 80 min; X axis). Band intensities on MWA blots were quantified using Li-cor Odyssey analysis software (Experimental Procedures). For each antibody, data was normalized to β-actin levels; error bars are the standard error of the mean of triplicate MWA blots. (C, D) 11 Plk-dependent phosphoproteins are critical for TLR-mediated antiviral responses in DCs. Shown are mRNA levels (qPCR; relative to non-targeting control shRNAs, Ctrl) for knockdown (KD) efficiency (left), Ifnb1 (middle), and Cxcl10 (right) in BMDCs following LPS stimulation. Genes with one and two shRNAs are shown in C and D, respectively. Three replicates in each experiment; error bars are the standard error of the mean. (E) Comparison of phosphosites identified in our study and in two recent reports (Weintz et al., and Sharma et al.). Shown are proportional Venn diagrams of the total unique phosphosites identified by the 3 studies (left), and the phosphosites harbored by kinases only (right). Total numbers of unique phosphosites per study are indicated in parentheses.

### DETAILED DESCRIPTION OF THE INVENTION

The invention is based upon the discovery that the polo-like kinase (PLK) family of proteins are signaling components of innate immune pathways. In particular, it was discovered that PLKs are co-opted by anti-viral pathways of dendritic cells and inhibition of PLKs impairs anti-viral gene induction in dendritic cells.

A perturbation strategy for reconstruction of regulatory networks was used to identify signaling components of the Toll-Like Receptor (TLR) that are transcriptionally regulated in dendritic cells. Regulatory networks controlling gene expression serve as decision-making circuits within cells. For example, when immune dendritic cells are exposed to viruses, bacteria, or fungi they responds with transcriptional programs that are specific to each pathogen and are essential for establishing appropriate immunological outcomes. However, altered functions of dendritic cells are also known to play a role in diseases such as allergy and autoimmune disease. Thus, identification of regulators in the innate immune pathway will allow therapeutic targeting of specific pathways to control disease.

Two hundred and eighty one (281) genes were found to be differentially regulated in TLR stimulated dendritic cells. Of these 281 genes, it was determined that the cell-cycle regulators polo-like kinase 2 and 4 (PLK) are anti-viral regulators. Inhibition of PLK using commercially available pan-specific PLK small molecule inhibitors resulted in a decrease in anti-viral gene expression in dendritic cells. Specifically, a decrease in IFN-b and CXCL10 mRNA expression in dendritic cells upon LPS stimulation. Accordingly, the invention provides methods of decreasing and/or treating inflammation associated with an innate immune response to a pathogen, e.g., virus, buy administering to a subject a polo-like kinase inhibitor. The invention also provides methods of decreasing anti-viral cytokine expression by contacting a dendritic cell with a PLK inhibitor.

### DEFINITIONS

Disease” or “disorder” refers to an impairment of the normal function of an organism. As used herein, a disease may be characterized by, e.g., an immune disorder, an inflammatory response, viral infection, bacterial infection or a combination of any of these conditions.

“Immune-modulating” refers to the ability of a compound of the present invention to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

“Immune disorder” refers to abnormal functioning of the immune system. Immune disorders can be caused by deficient immune responses (e.g., HIV AIDS) or overactive immune responses (e.g., allergy, auto-immune disorders). Immune disorders can result in the uncontrolled proliferation of immune cells, uncontrolled response to foreign antigens or organisms leading to allergic or inflammatory diseases, aberrant immune responses directed against host cells leading to auto-immune organ damage and dysfunction, or generalized suppression of the immune response leading to severe and recurrent infections.

“Dendritic cells” (DCs) are immune cells that form part of the mammalian immune system. Their main function is to process antigen material and present it on the surface to other cells of the immune system, thus functioning as antigen-presenting cells. They act as messengers between the innate and adaptive immunity.

“Innate immunity” refers to an early system of defense that depends on invariant receptors recognizing common features of pathogens. The innate immune system provides barriers and mechanisms to inhibit foreign substances, in particular through the action of macrophages and neutrophils. The inflammatory response is considered part of innate immunity. The innate immune system is involved in initiating adaptive immune responses and removing pathogens that have been targeted by an adaptive immune response. However, innate immunity can be evaded or overcome by many pathogens, and does not lead to immunological memory.

“Adaptive immunity” refers to the ability to recognize pathogens specifically and to provide enhanced protection against reinfection due to immunological memory based on clonal selection of lymphocytes bearing antigen-specific receptors. A process of random recombination of variable receptor gene segments and the pairing of different variable chains generates a population of lymphocytes, each bearing a distinct receptor, forming a repertoire of receptors that can recognize virtually any antigen. If the receptor on a lymphocyte is specific for a ubiquitous self antigen, the cell is normally eliminated by encountering the antigen early in its development. Adaptive immunity is normally initiated when an innate immune response fails to eliminate a new infection, and antigen and activated antigen-presenting cells are delivered to draining lymphoid tissues. When a recirculating lymphocyte encounters its specific foreign antigen in peripheral lymphoid tissues, it is induced to proliferate and its progeny then differentiate into effector cells that can eliminate the infectious agent. A subset of these proliferating lymphocytes differentiate into memory cells, capable of responding rapidly to the same pathogen if it is encountered again.

Immune disorders can be caused by an impaired or immunocompromised immune system can produce a deficient immune response that leaves the body vulnerable to various viral, bacterial, or fungal opportunistic infections. Causes of immune deficiency can include various illnesses such as viruses, chronic illness, or immune system illnesses. Diseases characterized by an impaired immune system include, but are not limited to, HIV AIDS and severe combined immunodeficiency syndrome (SCIDS).

Immune disorders caused by an excessive response by the immune system. This excessive response can be an excessive response to one or more antigens on a pathogen, or to an antigen that would normally be ignored by the immune system. Diseases characterized by an overactive immune system include, but are not limited to, arthritis, allergy, asthma, pollinosis, atopy, mucositis and auto-immune diseases. Anaphylaxis is a term used to refer an excessive immune system response that can lead to shock.

“Arthritis” refers to inflammation of the joints that can be caused, inter alia, by wear and tear on joints, or auto-immune attack on connective tissues, or exposure to an allergen, e.g., as in adjuvant-induced arthritis. Arthritis is often associated with, or initiated by, deposition of antibody-antigen complexes in joint membranes and activation of an inflammatory response. Sometimes the immune response is initiated by cells rather than antibodies, where the cells can produce a deposit in the joint membrane.

“Allergy” refers to an immune reaction to a normally innocuous environmental antigen (allergen), resulting from the interaction of the antigen with antibodies or primed T cells generated by prior exposure to the same antigen. Allergy is characterized by immune and inflammatory aspects, as the allergic reaction is triggered by binding of the antigen to antigen-specific IgE antibodies bound to a high-affinity IgE receptor on mast cells, which leads to antigen-induced cross-linking of IgE on mast cell surfaces, causing the release of large amounts of inflammatory mediators such as histamine. Later events in the allergic response involve leukotrienes, cytokines, and chemokines, which recruit and activate eosinophils and basophils. The late phase of this response can evolve into chronic inflammation, characterized by the presence of effector T cells and eosinophils, which is most clearly seen in chronic allergic asthma.

“Asthma” refers to a chronic inflammatory disorder affecting the bronchial tubes, usually triggered or aggravated by allergens or contaminants. Asthma is characterized by constriction of the bronchial tubes, producing symptoms including, but not limited to, cough, shortness of breath, wheezing, excess production of mucus, and chest constriction

“Atopy” refers to the tendency to develop so-called “classic” allergic diseases such as atopic dermatitis, allergic rhinitis (hay fever), and asthma, and is associated with a capacity to produce an immunoglobulin E (IgE) response to common allergens. Atopy is often characterized by skin allergies including but not limited to eczema, urticaria, and atopic dermatitis. Atopy can be caused or aggravated by inhaled allergens, food allergens, and skin contact with allergens, but an atopic allergic reaction may occur in areas of the body other than where contact with the allergan occurred. A strong genetic (inherited) component of atopy is suggested by the observation that the majority of atopic dermatitis patients have at least one relative who suffers from eczema, asthma, or hay fever. Atopy is sometimes called a “reagin response.”

“Mucositis’ is the painful inflammation and ulceration of the mucous membranes lining the digestive tract, usually as an adverse effect of chemotherapy and radiotherapy treatment for cancer. Mucositis can occur anywhere along the gastrointestinal (GI) tract, but oral mucositis refers to the particular inflammation and ulceration that occurs in the mouth. Oral mucositis is a common and often debilitating complication of cancer treatment.

“Pollinosis,” “hay fever,” or “allergic rhinitis,” are terms that refer to an allergy characterized by sneezing, itchy and watery eyes, a runny nose and a burning sensation of the palate and throat. Often seasonal, pollinosis is usually caused by allergies to airborne substances such as pollen, and the disease can sometimes be aggravated in an individual by exposure to other allergens to which the individual is allergic.

“Auto-immune” refers to an adaptive immune response directed at self antigens. “Auto-immune disease” refers to a condition wherein the immune system reacts to a “self” antigen that it would normally ignore, leading to destruction of normal body tissues. Auto-immune disorders are considered to be caused, at least in part, by a hypersensitivity reaction similar to allergies, because in both cases the immune system reacts to a substance that it normally would ignore. Auto-immune disorders include, but are not limited to, Hashimoto's thyroiditis, pernicious anemia, Addison's disease, type I (insulin dependent) diabetes, rheumatoid arthritis, systemic lupus erythematosus, dermatomyositis, Sjogren's syndrome, lupus erythematosus, multiple sclerosis, myasthenia gravis, Reiter's syndrome, and Grave's disease, alopecia greata, anklosing spondylitis, antiphospholipid syndrome, auto-immune hemolytic anemia, auto-immune hepatitis, auto-immune inner ear disease, auto-immune lymphoproliferative syndrome (ALPS), auto-immune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyneuropathy, cicatricial pemphigoid, cold agglutinin disease, CREST syndrome, Crohn's disease, Dego's disease, dermatomyositis, dermatomyositis, discoid lupus, essential mixed cryoglobulinemia, fibromyalgia-fibromyositis, Guillain-Barre syndrome, idiopathic pulmonary fibrosis, idiopathic thrombocytopenia purpura (ITP), IgA nephropathy, juvenile arthritis, Meniere's disease, mixed connective tissue disease, pemphigus vulgaris, polyarteritis nodosa, polychondritis, polyglancular syndromes, polymyalgia rheumatica, polymyositis, primary agammaglobulinemia, primary biliary cirrhosis, psoriasis, Raynaud's phenomenon, rheumatic fever, sarcoidosis, scleroderma, stiff-man syndrome, Takayasu arteritis, temporal arteritis/giant cell arteritis, ulcerative colitis, uveitis, vasculitis, vitiligo, and Wegener's granulomatosis.

“Inflammatory response” or “inflammation” is a general term for the local accumulation of fluid, plasma proteins, and white blood cells initiated by physical injury, infection, or a local immune response. Inflammation is an aspect of many diseases and disorders, including but not limited to diseases related to immune disorders, viral infection, arthritis, auto-immune diseases, collagen diseases, allergy, asthma, pollinosis, and atopy. Inflammation is characterized by rubor (redness), dolor (pain), calor (heat) and tumor (swelling), reflecting changes in local blood vessels leading to increased local blood flow which causes heat and redness, migration of leukocytes into surrounding tissues (extravasation), and the exit of fluid and proteins from the blood and their local accumulation in the inflamed tissue, which results in swelling and pain, as well as the accumulation of plasma proteins that aid in host defense. These changes are initiated by cytokines produced by activated macrophages. Inflammation is often accompanied by loss of function due to replacement of parenchymal tissue with damaged tissue (e.g., in damaged myocardium), reflexive disuse due to pain, and mechanical constraints on function, e.g., when a joint swells during acute inflammation, or when scar tissue bridging an inflamed joint contracts as it matures into a chronic inflammatory lesion.

“Anti-inflammatory” refers to the ability of a compound of the present invention to prevent or reduce the inflammatory response, or to soothe inflammation by reducing the symptoms of inflammation such as redness, pain, heat, or swelling.

Inflammatory responses can be triggered by injury, for example injury to skin, muscle, tendons, or nerves. Inflammatory responses can also be triggered as part of an immune response. Inflammatory responses can also be triggered by infection, where pathogen recognition and tissue damage can initiate an inflammatory response at the site of infection. Generally, infectious agents induce inflammatory responses by activating innate immunity. Inflammation combats infection by delivering additional effector molecules and cells to augment the killing of invading microorganisms by the front-line macrophages, by providing a physical barrier preventing the spread of infection, and by promoting repair of injured tissue. “Inflammatory disorder” is sometimes used to refer to chronic inflammation due to any cause.

Diseases characterized by inflammation of the skin, often characterized by skin rashes, include but are not limited to dermatitis, atopic dermatitis (eczema, atopy), contact dermatitis, dermatitis herpetiformis, generalized exfoliative dermatitis, seborrheic dermatitis, drug rashes, erythema multiforme, erythema nodosum, granuloma annulare, poison ivy, poison oak, toxic epidermal necrolysis and roseacae.

Inflammation can result from physical injury to the skin resulting in the “wheal and flare reaction” characterized by a mark at the site of injury due to immediate vasodilatation, followed by an enlarging red halo (the flare) due to spreading vasodilation, and elevation of the skin (swelling, the wheal) produced by loss of fluid and plasma proteins from transiently permeable postcapillary venules at the site of injury.

Inflammation triggered by various kinds of injuries to muscles, tendons or nerves caused by repetitive movement of a part of the body are generally referred to as repetitive strain injury (RSI). Diseases characterized by inflammation triggered by RSI include, but are not limited to, bursitis, carpal tunnel syndrome, Dupuytren's contracture, epicondylitis (e.g. “tennis elbow”), “ganglion” (inflammation in a cyst that has formed in a tendon sheath, usually occurring on the wrist) rotator cuff syndrome, tendinitis (e.g., inflammation of the Achilles tendon), tenosynovitis, and “trigger finger” (inflammation of the tendon sheaths of fingers or thumb accompanied by tendon swelling).

It is understood that the terms “immune disorder” and “inflammatory response” are not exclusive. It is understood that many immune disorders include acute (short term) or chronic (long term) inflammation. It is also understood that inflammation can have immune aspects and non-immune aspects. The role(s) of immune and nonimmune cells in a particular inflammatory response may vary with the type of inflammatory response, and may vary during the course of an inflammatory response. Immune aspects of inflammation and diseases related to inflammation can involve both innate and adaptive immunity. Certain diseases related to inflammation represent an interplay of immune and nonimmune cell interactions, for example intestinal inflammation (Fiocchi et al., 1997, Am J Physiol Gastrointest Liver Physiol 273: G769-G775), pneumonia (lung inflammation), or glomerulonephritis.

It is further understood that many diseases are characterized by both an immune disorder and an inflammatory response, such that the use of discrete terms “immune disorder” or “inflammatory response” is not intended to limit the scope of use or activity of the compounds of the present invention with respect to treating a particular disease. For example, arthritis is considered an immune disorder characterized by inflammation of joints, but arthritis is likewise considered an inflammatory disorder characterized by immune attack on joint tissues. In a disease having both immune and inflammatory aspects, merely measuring the effects of a compound of the present invention on inflammation does not exclude the possibility that the compound may also have immune-modulating activity in the same disease. Likewise, in a disease having both immune and inflammatory aspects, merely measuring the effects of a compound of the present invention on immune responses does not exclude the possibility that the compound may also have anti-inflammatory activity in the same disease.

“Viral infection” as used herein refers to infection of an organism by a virus that is pathogenic to that organism. It is understood that an infection is established after a virus has invaded tissues and then cells of the host organism, after which the virus has used the cellular machinery of the host to carry out functions that may include synthesis of viral enzymes, replication of viral nucleic acid, synthesis of viral packaging, and release of synthesized virus.

“Anti-viral” refers to the ability of a compound of the present invention to prevent, reduce, or eliminate a viral infection For example, an anti-viral compound of the invention may prevent viral attachment to cells, or viral entry, or viral uncoating, or synthesis of viral enzymes, or viral replication, or viral release. In particular, an anti-viral compound of the invention may prevent or otherwise inhibit viral replication in cells infected with the virus. An anti-viral compound of the invention may reduce (interfere with) viral attachment to cells, or viral entry, or viral uncoating, or synthesis of viral enzymes, or viral replication, or viral release, to such a degree that no significant disease (impairment of the normal function of an organism) results from the viral infection. An anti-viral compound of the invention may eliminate the viral infection by killing or weakening the virus so that it does not infect or replicate. An anti-viral compound of the invention may eliminate the viral infection through an immune-modulating effect that stimulates the immune system to kill the virus.

“Viral diseases,” “diseases characterized by viral infection,” and “diseases caused by viral infection” refer to impairment of the normal function of an organism as a result of viral infection. Diseases characterized by viral infection may include other aspects such as immune responses and inflammation. Compounds of the present invention are useful for treating diseases related to viral infection by RNA viruses, including retroviruses, or DNA viruses. A retrovirus includes any virus that expresses reverse transcriptase including, but not limited to, HIV-1, HIV-2, HTLV-I, HTLV-II, FeLV, FIV, SIV, AMV, MMTV, and MoMuLV.

Diseases related to viral infection can be caused by infection with a herpesvirus, arenavirus, coronavirus, enterovirus, bunyavirus, filovirus, flavivirus, hantavirus, rotavirus, arbovirus, Epstein-Barr virus, cytomegalovirus, infant cytomegalic virus, astrovirus, adenovirus and lentivirus, in particular HIV. Diseases related to viral infection (viral diseases) include, but are not limited to, molluscum contagiosum, HTLV, HTLV-1, HIV/AIDS, human papillomavirus, herpesvirus, herpes, genital herpes, viral dysentery, common cold, flu, measles, rubella, chicken pox, mumps, polio, rabies, mononucleosis, Ebola, respiratory syncytial virus (RSV), Dengue fever, yellow fever, Lassa fever, viral meningitis, West Nile fever, parainfluenza, chickenpox, smallpox, Dengue hemorrhagic fever, progressive multifocal leukoencephalopathy, viral gastroenteritis, acute Appendicitis, hepatitis A, hepatitis B, chronic hepatitis B, hepatitis C, chronic hepatitis C, hepatitis D, hepatitis E, hepatitis X, cold sores, ocular herpes, meningitis, encephalitis, shingles, pneumonia, encephalitis, California serogroup viral, St. Louis encephalitis, Rift Valley Fever, hand, foot, & mouth Disease, Hendra virus, Japanese encephalitis, lymphocytic choriomeningitis, roseola infantum, sandfly fever, SARS, warts, cat scratch disease, slap-cheek syndrome, orf, and pityriasis rosea.

It is understood that the terms “inflammatory response” and “viral infection” and “immune disorder” are not exclusive. Many diseases related to viral infection include inflammatory responses, where the inflammatory responses are usually part of the innate immune system triggered by the invading virus. Inflammation can also be triggered by physical (mechanical) injury to cells and tissues resulting from viral infection. Examples of viral infections characterized by inflammation include, but are not limited to: encephalitis, which is inflammation of the brain following viral infection with, e.g., arbovirus, herpesvirus, and measles (before vaccines were common); meningitis, which is inflammation of the meninges (the membranes that surround the brain and spinal cord) following infection; meningoencephalitis, which is infection and inflammation of both the brain and meninges; encephalomyelitis which is infection and inflammation of the brain and spinal cord; viral gastroenteritis, which is an inflammation of the stomach and intestines caused by a viral infection; viral hepatitis, which is an inflammation of the liver caused by viral infection.

Polo-Like Kinase Inhibitors

A polo like kinase (PLK) inhibitor is a compound that decreases expression or activity of one or more PLKs. A decrease in PLK expression or activity is defined by a reduction of a biological function of the PLK protein. PLKs include PLK1, PLK2, PLK3 and PLK4. PLKs are serine theronine protein kinases that are involved in the regulation of the cell cycle.

PLK expression is measured by detecting a PLK transcript or protein. PLK inhibitors are known in the art or are identified using methods described herein. For example, a PLK inhibitor is identified by detecting a decrease in cell proliferation by mitotic arrest. Mitotic arrest is measure by methods known in the art such as staining α-tubulin and DNA to identify mitotic statges.

The PLK inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Suitable, PLK inhibitors useful in the methods of the invention includes those described in WO2006/018185, WO2007/095188, WO2008/076392, US2010/0075973, US 2010/004250 and U.S. Pat. No. 6,673,801. Preferably, the PLK inhibitor is BI-2536 (Current Biology, Volume 17, Issue 4, 316-322, 20 Feb. 2007; CAS#755038-02-9); poloxipan (CAS #1239513-63-3); poloxin (Chemistry & Biology, Volume 15, Issue 5, 415-416, 19 May 2008; CAS#321688-88-4) Thymoquinone, or GW843682X (5-(5,6-Dimethoxy-1H-benzimidazol-1-yl)-3-[[2-(trifluoromethyl)phenyl]methoxy]-2-thiophenecarboxamide; CAS#2977; Lansing et al (2007) In vitro biological activity of a novel small-molecule inhibitor of polo-like kinase 1. Mol. Cancer Ther. 6 450.) The contents of each are hereby incorporated by reference in there entirety.

The PLK inhibitor is BI-2536, which is represented by Formula I below:

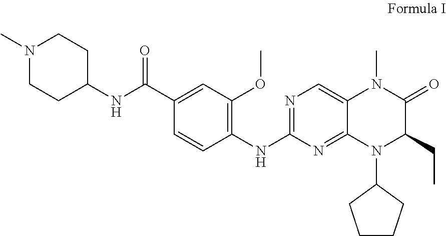 [SMILES: CC[C@@H]1C(=O)N(C)c2cnc(Nc3ccc(C(=O)NC4CCN(C)CC4)cc3OC)nc2N1C1CCCC1]

The PLK inhibitor is poloxipan, which is represented by Formula II below:

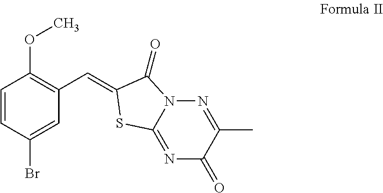 [SMILES: COc1ccc(Br)cc1/C=c1\sc2nc(=O)c(C)nn2c1=O]

The PLK inhibitor is GW843682X, which is represented by Formula III below:

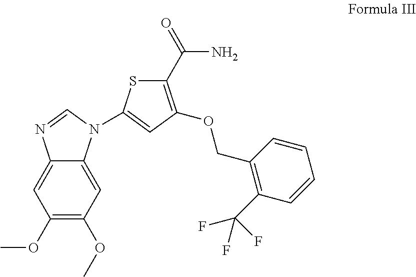 [SMILES: COc1cc2ncn(-c3cc(OCc4ccccc4C(F)(F)F)c(C(N)=O)s3)c2cc1OC]

The PLK inhibitor is poloxin, which is represented by Formula IV below:

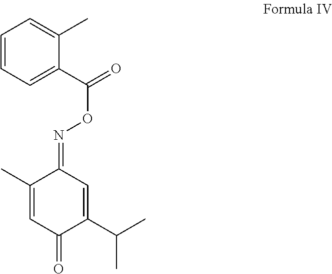 [SMILES: CC1=CC(=O)C(C(C)C)=C/C1=N\OC(=O)c1ccccc1C]

The PLK inhibitor is thymoquinone, which is represented by Formula V below:

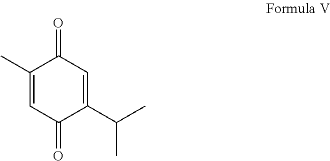

Other suitable PLK inhibitors useful in the methods of the invention include for example, cyclapolin, DAP-81, ZK-thiazolidinone, Compound 36, and LFM-A13.

Alternatively, the PLK inhibitor is for example an antisense PLK nucleic acid, a PLK-specific short-interfering RNA, or a PLK-specific ribozyme. By the term “siRNA” is meant a double stranded RNA molecule which prevents translation of a target mRNA. Standard techniques of introducing siRNA into a cell are used, including those in which DNA is a template from which an siRNA RNA is transcribed. The siRNA includes a sense PLK nucleic acid sequence, an anti-sense PLK nucleic acid sequence or both. Optionally, the siRNA is constructed such that a single transcript has both the sense and complementary antisense sequences from the target gene, e.g., a hairpin.

Binding of the siRNA to a PLK transcript in the target cell results in a reduction in PLK production by the cell. The length of the oligonucleotide is at least 10 nucleotides and may be as long as the naturally-occurring PLK transcript. Preferably, the oligonucleotide is 19-25 nucleotides in length. Most preferably, the oligonucleotide is less than 75, 50, 25 nucleotides in length.

The PLK inhibitor is specific for at least two PLKs (i.e., PLK1, PLK2, PLK3, PLK4). Preferably, the PLK inhibitor is a pan-specific PLK inhibitor. Most preferably, the PLK inhibitor is specific for at least PLK2 and PLK4.

Therapeutic Methods

The invention further provides a method of decreasing and or treating inflammation subject by administering the subject a PLK inhibitor. The inflammation is associated with an innate immune response to a pathogen or a pathogen derived molecule. The pathogen binds a toll-like receptor on the surface of a dendritic cell, or in endosomes. Alternatively, the pathogen bins cytosolic RIG-1-like recpetors such as for example RIG-1, MDA-5 of a dentritic cell. The pathogen is preferably a virus. Also provided are methods of decreasing anti-viral cytokine expression in a subject by administering to a subject in need thereof a Plk inhibitor. The cytokine is for example interferon-β or CXCL-10.

Efficaciousness of treatment is determined in association with any known method for diagnosing or treating the particular inflammatory disorder. Alleviation of one or more symptoms of the inflammatory disorder indicates that the compound confers a clinical benefit.

The invention further provides pharmaceutical compositions including a PLK inhibitor that can be administered to achieve a desired effect. The pharmaceutical composition includes at least one PLK inhibitor and a pharmaceutically acceptable carrier or excipient, and may optionally include additional ingredients.

The compounds of the invention can be administered systemically, regionally (e.g., directed towards an organ or tissue), or locally (e.g., intracavity or topically onto the skin), in accordance with any protocol or route that achieves the desired effect. The compounds can be administered as a single or multiple dose each day (e.g., at a low dose), or intermittently (e.g., every other day, once a week, etc. at a higher dose). The compounds and pharmaceutical compositions can be administered via inhalation (e.g., intra-tracheal), oral, intravenous, intraarterial, intravascular, intrathecal, intraperitoneal, intramuscular, subcutaneous, intracavity, transdermal (e.g., topical), or transmucosal (e.g., buccal, vaginal, uterine, rectal, or nasal) delivery. The pharmaceutical compositions can be administered in multiple administrations, by sustained release (e.g., gradual perfusion over time) or in a single bolus.

The term “subject” refers to animals, typically mammalian animals, such as primates (humans, apes, gibbons, chimpanzees, orangutans, macaques), domestic animals (dogs, cats, birds), farm animals (horses, cattle, goats, sheep, pigs) and experimental animals (mouse, rat, rabbit, guinea pig). Subjects include animal disease models. In some embodiments, the subject does not have cancer, has never had cancer, or has not been treated for cancer. For example, in some embodiments the subject has never received a PLK inhibitor to treat cancer.

Amounts administered are typically in an “effective amount” or “sufficient amount” that is an amount sufficient to produce the desired affect. Effective amounts are therefore amounts that induce PLK inhibition and one or more of: inhibiting or reducing susceptibility to inflammation, auto-immune diseases, mucositis, Parkinson's Disease, decreasing one or more symptoms associated with inflammation or viral infection, inhibiting or reducing cytokine expression, preferably interferon-β or CXCL-1-, or decreasing one or more symptoms associated with viral infection.

Effective amounts can objectively or subjectively reduce or decrease the severity or frequency of symptoms associated with inflammation, auto-immune diseases, mucositis, Parkinson's Disease, or an associated disorder or condition. For example, an amount of a compound of the invention that reduces itching, inflammation, pain, discharge or any other symptom or associated condition is an effective amount that produces a satisfactory clinical endpoint. Effective amounts also include a reduction of the amount (e.g., dosage) or frequency of treatment with another medicament to treat inflammation, auto-immune diseases, mucositis, Parkinson's Disease, which is considered a satisfactory clinical endpoint.

Methods of the invention that lead to an improvement in the subject's condition or a therapeutic benefit may be relatively short in duration, e.g., the improvement may last several hours, days or weeks, or extend over a longer period of time, e.g., months or years. An effective amount need not be a complete ablation of any or all symptoms of the condition or disorder. Thus, a satisfactory clinical endpoint for an effective amount is achieved when there is a subjective or objective improvement in the subjects' condition as determined using any of the foregoing criteria or other criteria known in the art appropriate for determining the status of the disorder or condition, over a short or long period of time. An amount effective to provide one or more beneficial effects, as described herein or known in the art, is referred to as an “improvement” of the subject's condition or “therapeutic benefit” to the subject.

An effective amount can be determined based upon animal studies or optionally in human clinical trials. The skilled artisan will appreciate the various factors that may influence the dosage or timing required to treat a particular subject including, for example, the general health, age, or gender of the subject, the severity or stage of the disorder or condition, previous treatments, susceptibility to undesirable side effects, clinical outcome desired or the presence of other disorders or conditions. Such factors may influence the dosage or timing required to provide an amount sufficient for therapeutic benefit.

Screening Assays

The invention also provides a method of screening for regulatory and transcriptional networks controlling gene expression. The methods allow the mechanistic basis for pathogen specific responses to be determined. In particular, the invention provides a method for identifying genes or genetic elements associated with a pathogen specific response by contacting a dendritic cell with a toll-like receptor agonist and identifying genes or genetic elements whose expression is induced toll-like receptor agonist. The pathogen is a virus, a bacteria, a fungus or a parasite. Toll-like receptor agonists include for example, Pam3CSK4, lipopolysaccharide, polyinosinic: polycytidylic acid, gardiquimod, or CpG. By induced is meant that gene expression is modulated (upregulated or downregulated) due to agonist treatment. Gene expression is measured by methods know in the art. In various embodiments the method further includes perturbing expression of the induced gene or genetic element. This perturbation allows for network reconstruction of the regulatory or transcriptional networks controlling gene expression. For example, RNA expression of the induced genes is inhibited by using anti-sense olignucleotide, siRNA, shRNA, RNAi or any other method known to interfere or inhibit expression of a target gene.

### EXAMPLES

### Example 1

### General Methods

Cells and Mouse Strains

Bone marrow-derived DCs were generated from 6-8 week old female C57BL/6J mice, Crkl mutant mice (Jackson Laboratories), Plk2^{-/-} mice (Elan Pharmaceuticals), or Ifnar1^{-/-} mice (gift from K. Fitzgerald). Primary mouse lung fibroblasts (MLFs) were from C57BL/6J mice.

Viruses

Sendai virus (SeV) strain Cantell and Encephalomyocarditis virus (EMCV) strain EMC (ATCC), Newcastle disease virus (NDV) strain Hitchner B1 (gift from A. Garcia-Sastre), and vesicular stomatitis virus (VSV) strain Indiana (U. von Andrian), were used for infections. Influenza A virus strain A/PR/8/34 and ΔNS1 were grown in Vero cells, and virus titers from MLF supernatants was quantified using 293T cells transfected with a vRNA luciferase reporter plasmid.

mRNA isolation, qPCR, and microarrays Total or polyA+ RNA was extracted and reverse transcribed prior to qPCR analysis with SYBR Green (Roche) in triplicate with GAPDH for normalization. For microarray analysis, Affymetrix Mouse Genome 430A 2.0 Array were used.

Preparation of Dendritic Cells

Bone marrow-derived dendritic cells (BMDCs) were generated from 6-8 week old female C57BL/6J mice (Jackson Laboratories). Bone marrow cells were collected from femora and tibiae and plated at 10⁶ cells/mL on non-tissue culture treated petri dishes in RPMI-1640 medium (Gibco), supplemented with 10% FBS, L-glutamine, penicillin/streptomycin, MEM non-essential amino acids, HEPES, sodium pyruvate, β-mercaptoethanol, and murine GM-CSF (15 ng/mL; Peprotech) or human Flt3L (100 ng/mL; Peprotech). GM-CSF-derived BMDCs were used directly for all RNAi experiments. For all other experiments, floating cells from GM-CSF cultures were sorted at day 5 by MACS using the CD11c (N418) MicroBeads kit (Miltenyi Biotec). Sorted CD11c⁺ cells were used as GM-CSF-derived BMDCs, and plated at 10⁶ cells/mL and stimulated at 16 h post sorting. For Flt3L culture, floating cells were harvested at day 6-8 and used as Flt3L-derived BMDCs by plating them at 10⁶ cells/mL and stimulating 16 h later. For SILAC experiments, GM-CSF-derived BMDCs were grown in media containing either normal L-arginine (Arg-0) and L-lysine (Lys-0) (Sigma) or L-arginine 13C6-15N4 (Arg-10) and L-lysine 13C6-15N2 (Lys-8) (Sigma Isotec). Concentrations for L-arginine and L-lysine were 42 mg/L and 40 mg/L, respectively. The cell culture media, RPMI-1640 deficient in L-arginine and L-lysine, was a custom media preparation from Caisson Laboratories (North Logan, Utah) and dialyzed serum was obtained from SAFC-Sigma. We followed all standard SILAC media preparation and labeling steps as previously described (Ong and Mann, 2006).

### Preparation of Primary Lung Fibroblasts

Mouse lung fibroblasts (MLFs) were derived from lung tissue from 6-8 week old female C57BL/6J mice (Jackson Laboratories). MLFs were isolated as previously described (Tager et al., 2004). Briefly, lungs were digested for 45 min at 37° C. in collagenase and DNase I, filtered, washed, and cultured in DMEM supplemented with 15% FBS. Cells were used for experiments between passages 2 and 5.

Genetically Modified Mice

Bone marrow from Plk2^{-/-} mice and their wild-type littermates were obtained from Elan Pharmaceuticals (Inglis et al., 2009). Ifnar1^{-/-} mice on a C57BL/6J background were a gift from Kate Fitzgerald (originally from Jonathan Sprent based on Muller et al., 1994). Heterozygous Crkl^{+/-} mice on a C57BL/6J background were obtained from the Jackson Laboratory. Crkl^{+/-} C57BL/6J mice were crossed to wild-type Black Swiss mice from Taconic, as Crkl^{-/-} mice on a pure C57BL/6J genetic background have been reported to be embryonic lethal (Guris et al., 2001; Hemmeryckx et al., 2002). Heterozygous Crkl^{+/-} offspring were backcrossed to Crkl^{+/-} C57BL/6J mice to obtain Crkl^{-/-} mice. Mice were kept in a specific pathogen-free facility at MIT. Animal procedures were in accordance with National Institutes of Health Guidelines on animal care and use, and were approved by the MIT Committee on Animal Care (Protocol #0609-058-12).

Viruses

Sendai virus (SeV), strain Cantell, and Encephalomyocarditis virus (EMCV), strain EMC, were from ATCC. Newcastle disease virus (NDV), strain Hitchner B1 was from Aldolfo Garcia-Sastre (Mount Sinai School of Medicine), and vesicular stomatitis virus (VSV), strain Indiana was from Ulrich von Andrian (Harvard Medical School). Influenza A virus strain A/PR/8/34 and ΔNS1 were grown in Vero cells (which allow efficient growth of the ΔNS1 virus) in serum-free DMEM supplemented with 10% BSA and 1 mg/ml TPCK trypsin. Viral titers were determined by standard MDCK plaque assay. To measure the amount of VSV RNA present in infected tissues, we used previously reported qPCR primers: VSV Forward 5′-TGATACAGTACAATTATTTTGGGAC-3′, and VSV Reverse 5′-GAGACTTTCTGTTACGGGATCTGG-3′ (Hole et al., 2006). Viruses were handled according to CDC and NIH guidelines with protocols approved by the Broad Institutional Biosafety Committee.

Reagents

TLR ligands were from Invivogen (Pam3CSK4, ultra-pure E. coli K12 LPS, ODN 1585 CpG type A, and ODN 1668 CpG type B) and Enzo Life Sciences (poly(I:C)), and were used at the following concentrations: Pam3CSK4 (250 ng/mL), poly(I:C) (10 μg/mL), LPS (100 ng/mL), CpG-A (10 μg/mL), CpG-B (10 μg/mL). Heat-killed Listeria monocytogenes (HKLM) was from Invivogen. Polo-like kinase inhibitors were from Selleck (BI 2536; Steegmaier et al., 2007), Sigma (GW843682X, also known as compound 1 and GSK461364; Lansing et al., 2007), and Chembridge (Poloxipan; Reindl et al., 2009). SP 600125 (Jnk inhibitor) was from Enzo Life Sciences. Image-iT FX Signal Enhancer, DAPI, and Alexa Fluor Labeled Secondary Antibodies were obtained from Invitrogen. For immunofluorescence, antibodies against IRF3 (4302S) and NF-κB P65 (4764S) were obtained from Cell Signaling Technology. For cell viability assays, Alamar Blue was from Invitrogen and CellTiter-Glo from Promega.

Virus Titering of MLF Supernatant

293T cells were seeded and transfected with a vRNA luciferase reporter plasmid as previously described (Shapira et al., 2009). Briefly, at 24 h post-transfection, 10⁴ transfected reporter cells were re-seeded in white Costar plates. Supernatants from influenza-infected MLFs were added to reporter cells and incubated for 24 h. Reporter activity was measured with firefly luciferase substrate (Steady-Glo, Promega). Luminescence activity was quantified with the Envision Multilabel Reader (Perkin Elmer).

Cell Cycle Analysis

Cells were fixed in ethanol, washed, and stained for 30 min at room temperature (RT) with propidium iodide (100 μg/mL) prepared in PBS (calcium- and magnesium-free) supplemented with RNAse A (2 mg/mL; Novagen) and triton X-100 (0.1%). Samples were analyzed for DNA content using an Accuri C6 flow cytometer (Accuri) and data was processed using the FlowJo software (Treestar).

ELISA

Cell culture supernatants were assayed using a sandwich ELISA kit for mouse IFN-β (PBL Biomedical Laboratories).

mRNA Isolation

Total RNA was extracted with QIAzol reagent following the miRNeasy kit's procedure (Qiagen), and sample quality was tested on a 2100 Bioanalyzer (Agilent). RNA was reverse transcribed with the High Capacity cDNA Reverse Transcription kit (Applied Biosystems). For experiments with more than 12 samples, we harvested PolyA+ RNA in 96- or 384-well plates with the Turbocapture mRNA kit (Qiagen) and reverse transcribed with the Sensiscript RT kit (Qiagen).

qPCR Measurements

Real time quantitative PCR reactions were performed on the LightCycler 480 system (Roche) with FastStart Universal SYBR Green Master Mix (Roche). Every reaction was run in triplicate and GAPDH levels were used as an endogenous control for normalization.

shRNA Knockdowns

High titer lentiviruses encoding shRNAs targeting genes of interest were obtained from The RNAi Consortium (TRC; Broad Institute, Cambridge, Mass., USA) (Moffat et al., 2006). Bone marrow cells were infected with lentiviruses as described (Amit et al., 2009). For each gene of interest, we tested five shRNAs for knock down efficiency using qPCR of the target gene. We selected shRNAs with >75% knockdown efficacy. For combinatorial knockdown, two independent mixtures of two lentiviruses encoding validated shRNAs against Plk2 and 4, respectively, were used to infect bone marrow cells (two Plk2- and two Plk4-specific shRNAs were used to generate these mixtures). Lentivirus-infected cells were composed of ˜90% CD11c⁺ cells, which was comparable to sorted BMDCs and to our previous observations (Amit et al., 2009).

mRNA Measurements on nCounter

Details on the nCounter system are presented in full in (Geiss et al., 2008). We used a custom CodeSet constructed to detect a total of 128 genes (including 10 control genes whose expression remain unaffected by TLR stimulation) selected by the GeneSelector algorithm (Amit et al., 2009) as described below. 5×10⁴ bone marrow-derived DCs were lysed in RLT buffer (Qiagen) supplemented with 1% (3-mercaptoethanol. 10% of the lysate was hybridized for 16 hours with the CodeSet and loaded into the nCounter prep station followed by quantification using the nCounter Digital Analyzer following the manufacturer's instructions. To score target genes whose expression is significantly affected by shRNA perturbations, we used a fold threshold corresponding to a false discovery rate (FDR) of 2%. Heatmaps and distance matrix analyses were generated using the Gene-E software (http://www.broadinstitute.org/cancer/software/GENE-E/).

Custom Nanostring CodeSet Construction Using the GeneSelector Algorithm

We used the CodeSet that we previously used and described in Amit et al., 2009. Briefly, to choose a set of genes that will capture as much as possible of the information on the expression of all genes, we used an information-theoretic approach. We modeled the expression levels X given the experimental condition C with a naive Bayes model where the expression of gene i under condition c follows a normal distribution Xᵢ|C=c˜N(μ_{ic},σᵢ²). In this model, the expression levels of all genes depend on the experimental condition C, and we selected genes that are highly informative about C. Formally, for a set of genes Y we used the conditional entropy H(C|Y)=−Σ_{c}p(C=c)Σ_{y}p(Y=y|C=c)log p(C=c|Y=y) as a measure of the remaining uncertainty in C once the expression levels Y are known. We then used this measure and a greedy procedure to select multiple disjoint gene sets, Y₁, . . . , Yₖ such that for each set Yᵢ, H(C|Yᵢ)<η (we set η=0.5). In the greedy procedure, we select genes one at a time, and with each selected gene re-compute the entropy given the genes already selected in the current set. Once a set is complete (the remaining conditional entropy is below the threshold η), we add all the genes to the selected set, and repeat the procedure (excluding all the selected genes from consideration). We stop when the number of selected genes has reached a user-defined threshold, set by the number of genes feasible for the experimental assay. To select a time point, we used the same approach. Here, we measured entropy under all time points for multiple randomly selected gene sets of several sizes and plotted the average entropy for each timepoint (see Amit et al., 2009). We chose the time point with the minimal entropy (i.e., 6 h post-simulation).

nCounter Data Analysis

After normalization by internal Nanostring controls (spike-normalization following manufacturer's instructions), we normalized the data relying on three control genes (Ndufa7, Tbca, Tomm7) that are the least affected by shRNAs and LPS stimulation. Next, we log-transformed the expression values (Bengtsson and Hossjer, 2006). Five signature genes (Cxcl5, Fos, Fst, Ereg, and Egr2) that were highly variable across control shRNA samples were removed from subsequent analysis. To score target genes whose expression is significantly affected by perturbations, we used a fold threshold corresponding to a false discovery rate (FDR) of 2%. For a given shRNA perturbation, a target gene was called as significantly affected when the ratio of the log-expression of this gene upon shRNA knockdown to the average log-expression of this gene in control shRNA samples was below (or above) a threshold (1/threshold), chosen such that, on average, no more than 2 hits (out of 128 genes in the Nanostring codeset) per control shRNA sample were called. Heatmaps and distance matrix analyses were generated using the software Gene-E (http://www.broadinstitute.org/cancer/software/GENE-E/)

Microarray Hybridization and Processing

For oligonucleotide microarray hybridization, 1 μg of RNA were labeled, fragmented, and hybridized to an Affymetrix Mouse Genome 430A 2.0 Array. After scanning, the expression value for each gene was calculated with RMA (Robust Multi-Array) normalization. The average intensity difference values were normalized across the sample set. Probe sets that were absent in all samples according to Affymetrix flags were removed. All values below 50 were floored to 50.

Detection of Regulated Signaling Genes

To identify differentially regulated signaling components (i.e., kinases, phosphatases, and signaling adaptors or scaffolds), we defined regulated probesets for each condition (TLR agonist) as probesets displaying at least 1.7-fold up- or down-regulation in both duplicates of at least one time point, compared to unstimulated controls, using our previously published microarray dataset available in the NCBI Gene Expression Omnibus under the accession number GSE17721 (Amit et al., 2009). Differentially regulated probesets were intersected with lists of kinases, phosphatases, and signaling adaptors and scaffolds. These gene sets were generated combining data from publicly available databases: Panther (http://www.pantherdb.org), Gene Ontology (http://www.geneontology.org), and DAVID (http://david.abcc.ncifcrf.gov). Regulated signaling genes were hierarchically clustered using the Cluster software (Eisen et al., 1998).

Antiviral Versus Inflammatory Gene Enrichment

Genes whose expression changed upon BI 2536 treatment in microarrays were evaluated for their enrichment with genes involved in the antiviral and inflammatory programs. When multiple probesets were available for a given gene on the microarray, only the probeset with maximum value was kept for analysis. Thus, the complete microarray consisted of 14088 genes, among which 804 and 550 genes were identified as part of antiviral and inflammatory programs, respectively (Amit et al., 2009). We performed a hypergeometric test on genes whose expression changed at least 3-fold upon BI 2536 treatment compared to vehicle control (DMSO), in either LPS or poly(I:C) samples. In addition, genes whose expression changed upon BI 2536 treatment in microarrays in response to LPS and/or poly(I:C) stimulation were analysed for enrichment of Gene Ontology (GO) processes and canonical pathways from curated databases using the Molecular Signature Databse (MSigDB; http://www.broadinstitute.org/gsea/msigdb/index.jsp).

Nanowire-Mediated Drug Delivery and Microscopy

BMDCs were plated on top of etched silicon nanowires (Si NWs) coated with small molecules (Shalek et al., 2010). After 24 hours, cells were stimulated with LPS or poly(I:C), and then fixed in 4% formaldehyde in PBS (RT, 10 min). After fixation, each sample was permeabilized with 0.25% Triton-X 100 in PBS (RT, 10 min), incubated with Image-iT FX Signal Enhancer (RT, 30 min), and then blocked with 10% goat serum and 0.25% Triton-X 100 in PBS (RT, 1 hour). After washing, the samples were placed in 3% IgG-Free BSA & 0.25% Triton-X 100 in PBS that contained primary antibodies against either IRF3 or NF-κB P65 (1:175 dilution) and then rocked overnight at 4° C. The following day, the samples were washed with PBS and then incubated with an Alexa Fluor labeled secondary antibody (1:250 dilution) in 3% IgG-Free BSA & 0.25% Triton-X 100 in PBS (RT, 60 min). After washing with PBS, the samples were counterstained with 300 ng/mL of DAPI in PBS (RT, 30 min). For each experiment, every stimulus-molecule combination was prepared in triplicate. Once fixed, samples were imaged using an upright confocal microscope (Olympus). For each captured image, the nuclear fraction of the fluorescent protein was calculated after identifying nuclear boundaries using DAPI. Finally, distributions for this quantity across different conditions were compared using a one-way ANOVA analysis.

In Vivo BI 2536 Experiments in a VSV Infection Model

8-week old C57BL/6 male mice (from Charles River Laboratories) received 500 μg of BI 2536 (or vehicle) intravenously, and 50 μg into the footpad 3 hours before and 2 hours after infection with 10⁶ pfu of VSV, as previously described (Iannacone et al., 2010), into the footpad. Mice were sacrificed 6 hours post-infection and the draining popliteal lymph nodes were harvested in RNAlater solution (Ambion) before subsequent RNA analysis. All experimental animal procedures were approved by the Institutional Animal Committees of Harvard Medical School and IDI. All infectious work was performed in designated BL2+ workspaces, in accordance with institutional guidelines, and approved by the Harvard Committee on Microbiological Safety.

MicroWestern Arrays

The MicroWestern Array (MWA) method previously described (Ciaccio et al., 2010) was modified to accommodate a larger number of lysates. The lysates were printed in a ‘double-block’ format with each MWA being 18 mm wide by 9 mm long. Twelve samples plus protein marker (Li-cor 928-40000) were printed with a non-contact piezoelectric arrayer (GeSiM NP2) along the top edge of the block, each block printed forty-eight times on the acrylamide gel. The deck layout is included in FIG. 14A. Electrophoresis, transfer, and antibody incubation were performed as previously described with the exception of using a modified 48-well gasket (The Gel Company MMH96) manually cut to have a larger block size in order to isolate antibodies on the nitrocellulose membrane per printed block. The antibodies used in this study were against β-ACTIN, GAPDH, β-TUBULIN, IκBα (clone L35A5), P65 (clone C22B4), STAT1, p-ABL(C−) (Y245), p-AKT (S473), p-AKT1/2/3 (T308), p-ATF2 (T71), p-ERK1/2 (T202/Y204), p-IKBALPHA (S32), p-IKKA/B (S176/180), p-IRF3 (S396), p-MAPKAPK2 (T222), p-MEK(1/2) (S217/221), p-MET (Y1234/1235), p-P38 (T180/Y182), p-P65 (S536), p-P70S6K (S371), p-P70S6K (T389), p-P90RSK (S380), p-PI3K P85(Y458) P55(Y199), p-PKCD (Y311), p-SAPK/JNK (T183/Y185), p-SEK1/MKK4 (T261), p-STAT1 (S727), p-STAT1 (Y701), p-STAT3 (S727). All antibodies were from Cell Signaling Technology, except for β-ACTIN which was from Santa Cruz Biotechnology. Band intensities were quantified using Li-cor Odyssey analysis software (V3.0). Circles were applied to the appropriate band on the scanned image and the net intensity was calculated by subtracting the background intensity from the trimmed mean intensity of each band. The net intensity was divided by the average net intensities of β-actin to control for lysate protein concentration. Fold Change was then calculated in relation to time of inhibitor application (time zero).

Phosphotyrosine Peptide Analysis

Tyrosine-phosphorylated peptides were prepared using a PhosphoScan Kit (Cell Signaling Technology) as previously described (Rush et al., 2005). Briefly, 100 million cells were lysed in lysis buffer (20 mM HEPES, 25 mM sodium pyrophosphate, 10 mM beta-glycerophosphate, 9 M urea, 1 mM ortho-vanadate, 1 Roche Ser/Thr phosphatase inhibitor tablet) assisted by sonication on ice using Misonix S-4000 sonicator with five 30-second bursts at 4 watts. Lysates were pre-cleared by centrifugation for 15 min at 20,000 g.˜10 mg of total proteins from each SILAC label were mixed, reduced with 10 mM dithiothreitol and alkylated with 25 mM iodoacetamide. After 4-fold dilution 200 μg sequencing grade modified trypsin (Promega, V5113) was added in an enzyme to substrate ratio of 1:100. The total peptide mixtures were then desalted using a tC18 SepPak cartridge (Waters, 500 mg, W AT036790) and resuspended in IAP buffer (50 mM MOPS/NaOH pH 7.2, 10 mM Na2HPO4, 50 mM NaCl). Peptide immunoprecipitation was performed with protein-G agarose bead-bound anti-phosphotyrosine antibodies pY100. Peptides captured by phosphotyrosine antibodies were eluted under acidic conditions (0.15% trifluoroacetic acid). The IP eluate was analyzed by data-dependent LC-MS/MS using a Thermo LTQ-Orbitrap instrument.

Global Serine, Threonine, and Tyrosine Phosphorylation Analysis

Quantitative analysis of serine, threonine and tyrosine phosphorylated peptides was performed essentially as described (Villen and Gygi, 2008) with some modifications. After stimulation, cells were lysed for 20 min in ice-cold lysis buffer (8 M Urea, 75 mM NaCl, 50 mM Tris pH 8.0, 1 mM EDTA, 2 μg/ml Aprotinin (Sigma, A6103), 10 μg/ml Leupeptin (Roche, #11017101001), 1 mM PMSF, 10 mM NaF, 2 mM Na3VO4, 50 ng/ml Calyculin A (Calbiochem, #208851), Phosphatase inhibitor cocktail 1 (1/100, Sigma, P2850) and Phosphatase inhibitor cocktail 2 (1/100, Sigma, P5726). Lysates were precleared by centrifugation at 16,500 g for 10 min and protein concentrations were determined by BCA assay (Pierce). We obtained 3 mg total protein per label out of 30 million cells. Cell lysates were mixed in equal amounts per label and proteins were reduced with 5 mM dithiothreitol and alkylated with 10 mM iodoacetamide. Samples were diluted 1:4 with HPLC water (Baker) and sequencing-grade modified trypsin (Promega, V5113) was added in an enzyme to substrate ratio of 1:150. After 16 h digest, samples were acidified with 0.5% trifluoroacetic acid (final concentration). Tryptic peptides were desalted on reverse phase tC18 SepPak columns (Waters, 500 mg, WAT036790) and lyophilized to dryness. Peptides were reconstituted in 500 μl strong cation exchange buffer A (7 mM KH2PO4, pH 2.65, 30% MeCN) and separated on a Polysulfoethyl A column from PolyLC (250×9.4 mm, 5 μm particle size, 200 A pore size) using an Akta Purifier 10 system (GE Healthcare). We used an 80 min gradient with a 20 min equilibration phase with buffer A, a linear increase to 30% buffer B (7 mM KH2PO4, pH 2.65, 350 mM KCL, 30% MeCN) within 33 min, 100% B for 7 min and a final equilibration with Buffer A for 20 min. The flow rate was 3 ml/min and the sample was injected after the initial 20 min equilibration phase. Upon injection, 3 ml fractions were collected with a P950 fraction collector throughout the run. 60 fractions were collected of which 3-4 adjacent fractions were combined to obtain 12 samples. Pooling of SCX fractions was guided by the UV214-trace and fractions were combined starting where the first peptide peak appeared. The 12 samples were desalted with reverse phase tC18 SepPak columns (Waters, 100 mg, WAT036820) and lyophilized to dryness. SCX-separated peptides were subjected to IMAC (immobilized metal affinity chromatography) as described (Villen and Gygi, 2008). Briefly, peptides were reconstituted in 200 μl IMAC binding buffer (40% MeCN, 0.1% FA) and incubated for 1 h with 5 μl of packed Phos-Select beads (Sigma, P9740) in batch mode. After incubation, samples were loaded on C18 StageTips (Rappsilber et al., 2007), washed twice with 500 IMAC binding buffer and washed once with 50μl 1% formic acid. Phosphorylated peptides were eluted from the Phos-Select resin to the C18 material by loading 3 times 70 μl of 500 mM K2HPO4 (pH 7.0). StageTips were washed with 50 μl of 1% formic acid to remove phosphate salts and eluted with 80 μl of 50% MeCN/0.1% formic acid. Samples were dried down by vacuum centrifugation and reconstituted in 8 μl 3% MeCN/0.1% formic acid.

NanoLC-MS/MS Analysis

All peptide samples were separated on an online nanoflow HPLC system (Agilent 1200) and analyzed on a LTQ Orbitrap Velos (Thermo Fisher Scientific) mass spectrometer. 4 μl of peptide sample were autosampled onto a 14 cm reverse phase fused-silica capillary column (New Objective, PicoFrit PF360-75-10-N-5 with 10 μm tip opening and 75 μm inner diameter) packed in-house with 3 μm ReproSil-Pur C18-AQ media (Dr. Maisch GmbH). The HPLC setup was connected via a custom-made electrospray ion source to the mass spectrometer. After sample injection, peptides were separated at an analytical flowrate of 200 mL/min with an 70 min linear gradient (˜0.29% B/min) from 10% solvent A (0.1% formic acid in water) to 30% solvent B (0.1% formic acid/90% acetonitrile). The run time was 130 min for a single sample, including sample loading and column reconditioning. Data-dependent acquisition was performed using the Xcalibur 2.1 software in positive ion mode. The instrument was recalibrated in real-time by co-injection of an internal standard from ambient air (“lock mass option”) (Olsen et al., 2005). Survey spectra were acquired in the orbitrap with a resolution of 60,000 and a mass range from 350 to 1750 m/z. In parallel, up to 16 of the most intense ions per cycle were isolated, fragmented and analyzed in the LTQ part of the instrument. Ions selected for MS/MS were dynamically excluded for 20 s after fragmentation. For the second biological replicate analysis peptides observed to be regulated in the first analysis were loaded into a global parent mass inclusion list and 4 MS/MS scans were reserved for precursors from the inclusion list whereas 12 were performed on the most intense ions per duty cycle.

Identification and Quantification of Peptides and Proteins

Mass spectra were processed using the Spectrum Mill software package (Agilent Technologies) v4.0 b that includes in-house developed features for SILAC-based quantitation and phoshosite localization and also with the MaxQuant software package (version 1.0.13.13) (Cox and Mann, 2008), which was used in combination with a Mascot search engine (version 2.2.0, Matrix Science). For peptide identification in Spectrum Mill an International Protein Index protein sequence database (IPI version 3.60, mouse) was used which was reversed on-the-fly at search time. In MaxQuant a concatenated forward and reversed IPI protein sequence database (version 3.60, mouse) was queried. The mass tolerance for precursor ions and for fragment ions was set to 7 ppm and 0.5 Da, respectively. Cysteine carbamidomethylation was searched as a fixed modification, whereas oxidation on methionine, N-acetylation (Protein) and phosphorylation on serine, threonine or tyrosine residues were considered as variable modifications. The enzyme specificity was set to trypsin and cleavage N-terminal of proline was allowed. The maximum of missed cleavages was set to 3. For peptide identification the maximum peptide FDR was set to 1%. The minimum identification score was to 5 in Spectrum Mill and to 10 in MaxQuant. SILAC ratios were obtained from the peptide export table in Spectrum Mill and the evidence table in MaxQuant. Arginine to Proline conversion was determined to be 3.42% and 5.55% for both biological replicates, respectively. The conversion was calculated by defining Arg-10 as a fixed modification and by quantifying the ratio between peptides containing normal L-proline (Pro-0) and 13C5-15N1-labeled proline (Pro-6) with MaxQuant. Each peptide SILAC ratio was corrected for arginine to proline conversion by the formula r[c]=r[o]/((1−p)̂n), where r[c] is the corrected ratio, r[o] the observed ratio, p the conversion rate and n the number of proline residues per peptide. The median ratios of all non-phosphorylated peptides were used to normalize the M/L and H/L ratios of all phosphorylated peptides. To allow better peptide grouping, phosphosite localization information obtained from SpectrumMill and MaxQuant were further simplified. Probability scores greater or equal 0.75 were called fully localized and designated with (1.0), scores smaller 0.75 and greater or equal to 0.5 were called ambiguously localized and designated with (0.5), whereas scores smaller than 0.5 were called non-localized and the total number of phosphorylation sites per peptide was designated with an underscore after the peptide sequence. Median SILAC ratios of phosphopeptides for each experiment were calculated over all versions of the same peptide including different charge states and methionine oxidation states. The highest scoring versions of each distinct peptide were reported per experiment. Overlapping data between SpectrumMill and MaxQuant as well as between different biological replicates was analyzed for discrepancies by calculating the mean and standard deviation over all residuals for different ratios of the same phosphopeptide. Residuals were calculated by subtracting the two values for each phosphopeptide derived by SpectrumMill or MaxQuant as well as by two different biological replicates. All peptides were filtered from the data set that had residuals greater than 3 standard deviations distant from the mean as they were not reproducible between two biological replicates or between SpectrumMill and MaxQuant. Data derived from both software packages was combined and MaxQuant data was reported when the same phosphopeptide was identified and quantified by both programs. Log 2 phosphopeptide ratios of BI-2536 treated vs untreated dendritic cells followed a normal distribution that was fitted using least squares regression. Mean and standard deviation values derived from the Gaussian fit were used to calculate p-values. An FDR-based measure was used to assess significance of the findings (Storey and Tibshirani, 2003).

### Example 2

### Transcripts for Signaling Components are Regulated Upon TLR Stimulation

To discover new components of pathogen-sensing pathways, we used genome-wide mRNA profiles, previously measured at 10 time points along 24 hours following stimulation of primary bone marrow-derived DCs (BMDCs) with lipopolysaccharide (LPS; TLR4 agonist), polyinosinic:polycytidylic acid (poly(I:C); recognized by TLR3 and the cytosolic viral sensor MDA-5), or Pam3CSK4 (PAM; TLR2 agonist) (Amit et al., 2009). These three TLRs activate transcriptional programs referred to here as “inflammatory” (TLR2), “antiviral” (TLR3), or both (TLR4) (FIG. 1A) (Amit et al., 2009; Doyle et al., 2002).

Our analysis uncovered 280 genes annotated as known or putative signaling molecules that were differentially expressed following stimulation: 115 kinases, 69 phosphatases, and 96 other regulators, such as adaptors and scaffolds (FIG. 1B and Example 1). These 280 genes were enriched for canonical pathways of the TLR network such as MAP kinase (P<1.22×10⁻¹⁵, overlap 25/87, hypergeometric test), TLR (e.g., Myd88, Traf6, Irak4, Tbk1; P<8.43×10^{-12, 21/86}), and PI3K (P<2.58×10⁻⁸, 11/33) pathways, as well as the PYK2 pathway (P<3.12×10⁻¹°, 12/29), which was recently associated with the TLR system (Wang et al., 2010). Overall, 94 of the 280 genes (33%) were associated with the TLR network in the literature, supporting the validity of our candidate selection strategy. The remaining 186 genes (67%) represent candidate TLR components. To test their putative function in TLR signaling, we selected a subset of 23 candidates based on their strong differential expression, and to proportionally represent the five main induced expression clusters (FIGS. 1B and 1C). We also selected 6 canonical TLR components (Myd88, Mapk9, Tbk1, Ikbke, Tank, and Map3k7) as benchmarks (FIGS. 1A and 1D).

### Example 3

### A Perturbation Strategy Places Novel Signaling Components within the Antiviral and Inflammatory Pathways

We perturbed our 6 positive controls and 17 of the 23 candidates in BMDCs using shRNA-encoding lentiviruses (six candidates showed poor knockdown efficiency). We stimulated the cells with LPS, and measured the effect of gene silencing on the mRNA levels of 118 TLR response signature genes, representing the inflammatory and antiviral programs, using a multiplex mRNA counting method (FIG. 2A). Notably, the expression of the 118-genes was not affected in BMDCs transduced with lentivirus compared to untransduced cells (Amit et al., 2009). We determined statistically significant changes in the expression of signature transcripts upon individual knockdowns based on comparison to 10 control genes, whose expression remains unchanged upon TLR activation, and to control shRNAs (Experimental Procedures). Finally, we associated signaling molecules and downstream transcriptional regulators that may act in the same pathway by comparing the perturbational profiles of the 23 signaling molecules (6 canonical and 17 candidates) to each other and to those of the 123 transcription regulators previously tested (FIG. 2 and FIG. 9) (Amit et al., 2009).

Perturbing 5 of the 6 positive control signaling molecules strongly affected the expression of TLR signature genes, consistent with their known roles (FIG. 2A) and validating our approach. For example, perturbing Myd88, a known inflammatory adaptor, specifically abrogated the transcription of inflammatory genes (e.g., Cxcl1, Il1a, Il1b, Ptgs2, Tnf; FIG. 2A), similar to perturbations of downstream inflammatory transcription factors (e.g., Nfkb1, Nfkbiz; FIG. 2B). In addition, Tank acted as a negative regulator of a subset of antiviral genes (FIG. 2A), as expected (Kawagoe et al., 2009), and Tbk1 knockdown affected both antiviral and inflammatory outputs (FIG. 2A), consistent with findings that Tbk1 regulates NF-κB complexes (Barbie et al., 2009; Chien et al., 2006). Notably, Ikbke (IKK-ε) knockdown did not affect our gene signature, consistent with previous observations that IKK-ε^{-/-} DCs respond normally to LPS and viral challenges (Matsui et al., 2006). Thus, IKK-c may either be not functional or redundant in our system.

All of the 17 candidate signaling molecules tested, except Plk2 (discussed below), affected at least 6 of the 118 genes (on average, 16.6 targets±10.4SD), and 12 affected more than 10% of the genes (FIGS. 9A and 9D). Notably, perturbations of these 17 candidates did not affect BMDC differentiation (88.3%±6.8 SD of CD11c⁺ cells). These effects are comparable to those of known signaling molecules and transcriptional regulators in this system (FIG. 9B-E). For example, the receptor tyrosine kinase Met, not previously associated with TLR signaling, affected a number of signature genes similar to Tbk1 (FIGS. 9C and 9D), in both the inflammatory and antiviral programs (FIG. 2A). Conversely, both the phosphatase Ptpre and the adaptor Socs6 positively regulated the inflammatory program, while negatively regulating some antiviral genes (FIG. 2B). Of the 17 candidates tested when we originally conducted this screen, 10 have subsequently been reported by others as functional in the TLR system, providing an independent confirmation. For example, Map3k8 knockdown affected here both inflammatory and antiviral target genes (FIG. 2A), consistent with its reported role in the TLR pathways based on Sluggish mice (Xiao et al., 2009).

We identified both primary (e.g., Myd88) and secondary (e.g., Stat1) mediators of TLR responses. While secondary mediators are not part of the initial intracellular signaling cascade, they are important physiological components of the TLR response and their pertubation can lead to similar phenotypic outcomes as that of primary components. For example, the receptor tyrosine kinase Mertk acted as both a positive and negative regulator of some inflammatory and antiviral genes (e.g., Ifnb1) respectively (FIG. 2A), consistent with its reported role as a secondary inhibitor of the TLR pathways (Rothlin et al., 2007).

### Example 4

### Crkl Modulates JNK-Mediated Antiviral Signaling in the TLR Network

Among the 17 candidate signaling proteins, perturbation of the tyrosine kinase adaptor Crkl decreased expression of 13% of the signature genes, especially antiviral ones (FIG. 2A and FIG. 9D). Crkl belongs to several signaling pathways, including early lymphocyte activation (Birge et al., 2009), but has not been associated with the TLR network. Crkl's perturbation profile closely resembled those of known antiviral regulators, most notably Jnk2 (Mapk9; Chu et al., 1999) (FIGS. 2A and 3A). Indeed, when Crkl^{-/-} DCs were stimulated with LPS, the expression of antiviral cytokines (Cxcl10, Ifnb1) was strongly reduced (FIG. 3B, left and middle), but that of an inflammatory cytokine (Cxcl1) was unaffected (FIG. 3B, right).

To test whether Crkl is a primary component of the TLR pathway, we measured if Crkl phosphorylation is rapidly modified after TLR signaling initiation. Using SILAC-based (Ong et al., 2002) quantitative phosphoproteomics, we identified and quantified 62 phospho-tyrosine (pTyr)-containing peptides from BMDCs stimulated with LPS for 30 minutes (FIG. 3C and Example 1). Of these 62 phosphopeptides, 7 and 9 were significantly up- or down-regulated, respectively (FIG. 3C). A phosphopeptide derived from Crkl (Y132)—one of the top-six induced phosphopeptides—was induced 2.1 fold (FIG. 3C). This indicates that Crkl is likely activated directly downstream of TLR4 signaling.

Several lines of evidence suggest that Crkl acts through Jnk2 (Mapk9) signaling. First, the MAP kinase Jnk2 (Mapk9) is co-regulated at the phosphorylation level with Crkl upon LPS stimulation (FIG. 3C). Second, the Crk adaptor family—including CrkI, CrkII, and Crkl—has been shown to modulate Jnk activity in growth factor and IFN signaling (Birge et al., 2009; Hrincius et al., 2010). Third, the perturbation profiles of Mapk9 and Crkl are strikingly similar (FIG. 3A). These observations suggest that Crkl modulates Jnk-mediated antiviral signaling in the TLR4 pathway, providing a possible explanation for why the NS1 protein of influenza A virus may target Crkl (Heikkinen et al., 2008; Hrincius et al., 2010).

### Example 5

### Polo-Like Kinases are Critical Activators of the Antiviral Program

To discover potential drug targets among our 17 candidates, we next focused on Polo-like kinase (Plk)2, a well-known cell cycle regulator and drug target (Strebhardt, 2010). The roles of Plks in non-dividing, differentiated cells are poorly defined (Archambault and Glover, 2009; Strebhardt, 2010). We have previously shown that transcriptional regulators of cell cycle processes (e.g., Rbl1, Rb, Myc, Jun, E2fs) are co-opted to function in the antiviral responses in DCs (Amit et al., 2009). However, neither knockdown (FIG. 2A) nor knockout (FIG. 10A) of Plk2 in BMDCs had any effect on the TLR response. We hypothesized that this could be due to functional redundancy with another Plk, since Plk4 mRNA was induced in DCs similarly to Plk2 (FIG. 4A), albeit at a lower amplitude (and thus was below our threshold for inclusion in the initial candidate list). Interestingly, functional redundancy between Plk2 and 4 has been suggested to account for the viability of Plk2-deficient mice (Strebhardt, 2010), and Plk2 and 4 have been reported to function together in centriole duplication (Chang et al., 2010; Cizmecioglu et al., 2008).

To test our hypothesis, we simultaneously perturbed Plk2 and 4 in BMDCs using two independent mixes of different pairs of shPlk2/shPlk4 (FIG. 10B and Example 1). We observed a significant and specific decrease in the expression of 21 antiviral genes (FIG. 4B). For example, the antiviral cytokines Ifnb1 and Cxcl10 mRNAs were decreased, whereas the expression of the inflammatory gene Cxcl1 and almost all inflammatory signature genes remained unaffected (FIG. 4C). Two recent reports suggested a role for Plk1 alone as a negative regulator of MAVS (Vitour et al., 2009) and NF-κB (Zhang et al., 2010) in cell lines. However, knockdown of either Plk1 or Plk3 in BMDCs did not affect the TLR transcriptional response (FIG. 10C). Notably, BMDC viability was unaffected by lentiviral shRNA transduction targeting Plk1, 2, 3 or 4 individually, or Plk2 and 4 together (based on mRNA levels of control genes). Thus, in BMDCs, Plk2 and 4, but likely not Plk1 or 3, are critical regulators of antiviral but not cell cycle pathways.

### Example 6

### A Small Molecule Inhibitor of Plks Represses Antiviral Gene Expression and IRF3 Translocation in DCs

We next targeted Plks in BMDCs using BI 2536, a commercial pan-specific Plk small molecule inhibitor (Steegmaier et al., 2007). We compared genome-wide mRNA profiles from BMDCs treated with either BI 2536 or DMSO vehicle before stimulation with LPS or poly(I:C) (Experimental Procedures). BI 2536 treatment repressed mostly antiviral gene expression compared to DMSO (99/193 genes in response to poly(I:C), P<1×10⁻⁷¹, hypergeometric test; 67/194 in response to LPS). The 311 unique LPS- and/or poly(I:C)-induced genes that are repressed by BI 2536, are significantly enriched for genes related to cytokine signaling (e.g., IL-10, type I IFNs, IL-1), TLR signaling, and DC signaling, and for GO processes related to defense and immune responses (FIG. 11A). Consistent with the array data, BI 2536 strongly inhibited the expression of 12 well-studied antiviral genes whereas inflammatory gene expression remained largely unaffected in DCs stimulated with LPS, poly(I:C), or Pam3CSK4, as measured by qPCR (FIG. 4D).

BI 2536 reduced the mRNA levels of Cxcl10 and Ifnb1 (by qPCR) and of secreted IFN-β in a dose-dependent manner, while Cxcl1 expression was not significantly affected (FIGS. 11B and 11C). Importantly, BI 2536 treatment pre-stimulation neither impacted the viability nor the cell cycle state of BMDCs (FIGS. 11D and 11E), suggesting that Plk inhibition does not act through cell cycle effects. Consistent with our shRNA and BI 2536 perturbations, two other pan-Plk inhibitors—structurally unrelated to BI 2536—also repressed Ifnb1 and Cxcl10 expression without affecting Cxcl1 (FIG. 11F). This strongly suggests that the effects induced by these perturbations are due to Plks inhibition, and not off-target effects. Furthermore, we observed a similar inhibitory effect of BI 2536 on Ifnb1 induction in Ifnar1^{-/-} and wild-type BMDCs, demonstrating that Plks act directly downstream of TLR activation, and not in an autocrine/paracrine feedback loop mediated by IFN receptor signaling (FIG. 11G). This is consistent with a recent phosphoproteomic study reporting an enrichment for Plk substrates as early as 15 min after LPS stimulation in macrophages (Weintz et al., 2010).

We next used confocal microscopy to monitor the effect of BI 2536 on the subcellular localization of IRF3, a key antiviral transcription factor. To more effectively deliver the drug, we plated BMDCs on vertical silicon nanowires (Shalek et al., 2010) pre-coated with BI 2536 pre-stimulation. Nanowires alone had no effect on the TLR response (FIG. 5A and FIG. 12A). BI 2536 inhibited IRF3 nuclear translocation in a dose-dependent manner upon poly(I:C) or LPS stimulation, whereas the control JNK inhibitor SP 600125 had no effect (FIGS. 5B and 5C, and FIG. 12B). On the other hand, BI 2536 did not affect NF-κB p65 localization (FIGS. 5D and 5E). Notably, IRF3 translocation was also decreased when delivering BI 2536 in solution, but to a lesser extent compared to nanowire-mediated delivery (FIG. 12C), highlighting the utility of highly efficient drug delivery methods to induce homogeneous effects in single-cell assays. Altogether, these results place Plk2 and 4 as critical regulators of the antiviral program, upstream of a major antiviral transcription factor.

### Example 7

### Plks are Essential for Activation of all Well-Established IFN-Inducing Pathways in Conventional and Plasmacytoid DCs

DCs can be broadly categorized into two major subtypes—conventional and plasmacytoid DCs—each relying on distinct mechanisms to induce type I IFNs and antiviral gene expression (Blasius and Beutler, 2010). In conventional DCs (cDCs), antiviral responses are activated through TLR4/3 signaling (via TRIF), or through the cytosolic sensors RIG-I or MDA-5 (via MAVS) (FIG. 6A). In plasmacytoid DCs (pDCs; specialized IFN-producing cells), the antiviral response depends solely on endosomal TLR7 and 9 that signal via MYD88 (FIG. 6A) (Blasius and Beutler, 2010; Takeuchi and Akira, 2010). BI 2536 treatment showed that Plks are essential for the viral-sensing pathways in both cDCs and pDCs. In cDCs, BI 2536 inhibited the transcription of antiviral genes (Ifnb1 and Cxcl10) upon infection with each of four viruses: vesicular stomatitis virus (VSV, FIG. 6B, top), Sendai virus (SeV; FIG. 13A top), or Newcastle disease virus (NDV; FIG. 13A bottom), all three sensed through RIG-I, and encephalomyocarditis virus (EMCV), sensed through MDA-5 (FIG. 6B, bottom and Example 1. Notably, BI 2536 neither affected the mRNA level of Cxcl1 (an inflammatory cytokine) in any of the four cases, nor affected the response to heat-killed Listeria monocytogenes, a natural TLR2 agonist (FIG. 6B and FIGS. 13A and 13B). In pDCs, BI 2536 treatment nearly abrogated the transcription of mRNAs for the antiviral cytokines Ifnb1, Ifna2, and Cxcl10 after stimulation with type A CpG oligonucleotides (CpG-A), or infection with EMCV, sensed by TLR9 and 7, respectively (FIG. 6C, FIG. 13C, and Example 1). Conversely, in pDCs stimulated with CpG-B—a ligand known to activate inflammatory pathways but not IFN-inducing pathways—BI 2536 treatment decreased Cxcl10 mRNA, while moderately increasing Cxcl1 mRNA (FIG. 6C). Finally, of our 118 signature genes, BI 2536 repressed genes induced by CpG-A alone or by both CpG-A and -B, while having a minor effect, if any, on CpG-B-specific genes in pDCs (FIG. 6D). These findings may help reveal the poorly characterized molecular determinants of IFN production in pDCs (Reizis et al., 2011), and demonstrate a critical role for Plks across all well-known IFN-inducing pathways.

### Example 8

### Plks are Essential in the Control of Host Antiviral Responses

To assess the impact of Plk inhibition on the outcome of viral infection, we infected primary mouse lung fibroblasts (MLFs) with influenza virus. BI 2536-treated MLFs infected with influenza failed to produce interferon (FIG. 6E), and showed elevated replication of both wild-type (PR8) and poorly-replicating mutant (ΔNS1) viruses (FIG. 6F). The reduced interferon response was not due to drug-induced toxicity (FIG. 6G).

Next, we tested the effects of Plk inhibition in virally infected mice. BI 2536 exhibits good tolerability in mice (Steegmaier et al., 2007) and humans (Mross et al., 2008), and is currently in Phase II clinical trials as an anti-tumor agent in several cancers (Strebhardt, 2010). Given its efficacy and safety in vivo, we tested whether BI 2536 would also affect the response to viral infection in animals. In mice infected with VSV, BI 2536 strongly suppressed13D). Concomitantly, VSV replication in the lymph node rapidly increased as reflected by elevated VSV RNA levels (FIG. 61), comparable to the observed phenotype of VSV-infected Ifnar1^{-/-} mice (Iannacone et al., 2010). Because in the VSV model used here type I IFNs are produced by both infected CD169⁺ subcapsular sinus macrophages and pDCs (Iannacone et al., 2010), we cannot distinguish whether Plk inhibition affects macrophages, pDCs, or both. Nevertheless, our results confirm the physiological importance of Plks in the host antiviral response in both ex vivo primary MLFs and in vivo mouse lymph nodes.

### Example 9

### Plks Affect the Phosphorylation of Dozens of Proteins Post-LPS Stimulation, Including Known Antiviral Components and Many Novel Components

We next sought to discover the signaling pathways between Plks and antiviral gene transcription. We used MicroWestern Arrays (MWAs) (Ciaccio et al., 2010) to measure changes in the phosphorylation and protein levels of 20 and 6 TLR pathway proteins, respectively, in BMDCs at each of 12 combinations of four time points (0, 20, 40, 80 min after LPS stimulation) and three perturbations (vehicle control, BI 2536, and negative control JNK inhibitor SP 600125). While LPS stimulation alone led to the expected changes (e.g., early peak of phosphorylation for ERK1/2, p38, and Mapkapk2, and rapid degradation of IκBα; FIG. 7A), BI 2536 surprisingly did cause any significant changes (FIG. 7A and FIGS. 14A and 14B). We therefore hypothesized that Plks could affect previously unrecognized regulators of IFN-inducing pathways and/or known regulators with no existing antibodies to specific phosphosites.

Next, we used SILAC-based unbiased phosphoproteomics (FIG. 7B top) (Villen and Gygi, 2008) to compared the levels of phospho-tyrosine, -threonine and -serine peptides following stimulation with LPS (for 30 or 120 min) in BMDCs pre-treated with BI 2536 versus those treated with vehicle (DMSO). We identified and quantified 5,061 and 5,997 phosphopeptides after 30 and 120 minutes, respectively, for a total of 10,236 individual phosphosites (FIG. 7B). BI 2536 substantially affected the TLR phosphoproteome, leading to a significant (P<0.001) change in the level of 510 phosphopeptides derived from 413 distinct proteins (FIG. 7B). Further supporting our results, 35% (2489/7018) of the phospho-sites we identified were recently reported in mouse bone marrow-derived macrophages treated with LPS (FIG. 14C, left) (Weintz et al., 2010), and 483 of our phosphosites were among 1858 sites (26%) reported in a phosphoproteomic study of LPS signaling in a macrophage cell line (FIG. 14C, left) (Sharma et al., 2010). A comparison of the phosphosites of known kinases showed similar overlaps between the three studies (FIG. 14C, right).

The Plk-dependent phosphoproteins include several known regulators of antiviral pathways (e.g., Prdm1, Fos, Unc13d) (Crozat et al., 2007; Keller and Maniatis, 1991; Takayanagi et al., 2002), as well as many additional protein candidates with no previously known function in viral sensing (FIG. 7B). Notably, proteins involved in the TBK1/IKK-c/IRF3 axis were detected and quantified, but their phosphorylation levels were unchanged upon Plk inhibiton, consistent with the MicroWestern array data. Conversely, Plk inhibition with BI 2536 decreased the phosphorylation levels of cell cycle regulators of the Jun family of transcriptional regulators (i.e., Jund) that we previously found to be co-opted by antiviral pathways (Amit et al., 2009). BI 2536 treatment also decreased the phosphorylation levels of the mitotic kinases Nek6 and Nek7 (FIG. 7B). The recent observation that the phosphorylation Nek6 substrates are increased following LPS stimulation in macrophages (Weintz et al., 2010) indirectly corroborates our finding that Nek6 may be active in TLR signaling. To test the role of these new candidates, we returned to our shRNA perturbation-based approach.

### Example 10

### Plk-Dependent Phosphoproteins Affect the Antiviral Response

We perturbed 25 Plk-dependent phosphoproteins, using shRNA perturbation in BMDCs followed by qPCR and TLR gene signature measurements. These candidates satisfied three criteria: (1) there was no prior knowledge of their function in viral sensing pathways; (2) their phosphoprotein levels were consistently up- or down-regulated upon BI 2536 treatment (in two independent experiments); and (3) they had detectable mRNA expression and/or differential expression upon stimulation.

Of the 18 phosphoproteins showing efficient knockdown, 11 caused a significant decrease in Ifnb1 mRNA levels with a single shRNA (Sash1, Dock8, Nek6, Nek7, Nfatc2, and Ankrd17; FIG. 14D), or with two independent shRNAs (Tnfaip2, Samsn1, Arhgap21, Mark2, and Zc3h14; FIG. 14E). Decrease in Cxcl10 expression was less prominent, consistent with our previous observations of BI2536's weaker effect on this cytokine during LPS stimulation (FIGS. 14D and 14E, far right panels). Each of the 11 Plk-dependent phosphoproteins tested affected at least 9 targets in the 118-gene signature (on average, 39 targets±30 SD; FIG. 7C), and 9 affected more than 10% of the targets in the TLR gene signature (FIG. 7C).

9 of the 11 Plk-dependent phosphoproteins affected the TLR signature comparably to major antiviral regulators (FIG. 7D). For example, the profiles of the newly identified candidates Samsn1, Dock8, and Sash1 were closely correlated to those of Stat and Irf family members (FIG. 7D), and those of that of Tnfaip2 and Zc3h14 were most correlated to the Plk2/4 double knockdown. Interestingly, Tnfaip2, a protein of unknown molecular function, has been associated with rheumatoid arthritis and autoimmune myocarditis in genome-wide association studies (Wellcome Trust Case Control Consortium, 2007; Kuan et al., 1999). Our findings provide a potential molecular context for this disease association.

### REFERENCES

Amit, I., Citri, A., Shay, T., Lu, Y., Katz, M., Zhang, F., Tarcic, G., Siwak, D., Lahad, J., Jacob-Hirsch, J., et al. (2007). A module of negative feedback regulators defines growth factor signaling. Nat Genet. 39, 503-512.
Amit, I., Garber, M., Chevrier, N., Leite, A. P., Donner, Y., Eisenhaure, T., Guttman, M., Grenier, J. K., Li, W., Zuk, O., et al. (2009). Unbiased reconstruction of a mammalian transcriptional network mediating pathogen responses. Science 326, 257-263.
Archambault, V., and Glover, D. M. (2009). Polo-like kinases: conservation and divergence in their functions and regulation. Nat Rev Mol Cell Biol 10, 265-275.
Banchereau, J., and Pascual, V. (2006). Type I interferon in systemic lupus erythematosus and other autoimmune diseases. Immunity 25, 383-392.
Barbie, D. A., Tamayo, P., Boehm, J. S., Kim, S. Y., Moody, S. E., Dunn, I. F., Schinzel, A. C., Sandy, P., Meylan, E., Scholl, C., et al. (2009). Systematic RNA interference reveals that oncogenic KRAS-driven cancers require TBK1. Nature 462, 108-112.
Barrat, F. J., and Coffman, R. L. (2008). Development of TLR inhibitors for the treatment of autoimmune diseases. Immunol Rev 223, 271-283.
Birge, R. B., Kalodimos, C., Inagaki, F., and Tanaka, S. (2009). Crk and CrkL adaptor proteins: networks for physiological and pathological signaling. Cell Commun Signal 7, 13.
Blasius, A. L., and Beutler, B. (2010). Intracellular toll-like receptors. Immunity 32, 305-315.
Chang, J., Cizmecioglu, O., Hoffmann, I., and Rhee, K. (2010). PLK2 phosphorylation is critical for CPAP function in procentriole formation during the centrosome cycle. EMBO J. 29, 2395-2406.
Chien, Y., Kim, S., Bumeister, R., Loo, Y. M., Kwon, S. W., Johnson, C. L., Balakireva, M. G., Romeo, Y., Kopelovich, L., Gale, M., Jr., et al. (2006). RalB GTPase-mediated activation of the IkappaB family kinase TBK1 couples innate immune signaling to tumor cell survival. Cell 127, 157-170.
Chu, W. M., Ostertag, D., Li, Z. W., Chang, L., Chen, Y., Hu, Y., Williams, B., Perrault, J., and Karin, M. (1999). JNK2 and IKKbeta are required for activating the innate response to viral infection. Immunity 11, 721-731.
Ciaccio, M. F., Wagner, J. P., Chuu, C. P., Lauffenburger, D. A., and Jones, R. B. (2010). Systems analysis of EGF receptor signaling dynamics with microwestern arrays. Nat Methods 7, 148-155.
Cizmecioglu, O., Warnke, S., Arnold, M., Duensing, S., and Hoffmann, I. (2008). Plk2 regulated centriole duplication is dependent on its localization to the centrioles and a functional polo-box domain. Cell Cycle 7, 3548-3555.
Cox, J., and Mann, M. (2008). MaxQuant enables high peptide identification rates, individualized p.p.b.-range mass accuracies and proteome-wide protein quantification. Nat Biotechnol 26, 1367-1372.
Crozat, K., Hoebe, K., Ugolini, S., Hong, N. A., Janssen, E., Rutschmann, S., Mudd, S., Sovath, S., Vivier, E., and Beutler, B. (2007). Jinx, an MCMV susceptibility phenotype caused by disruption of Unc13d: a mouse model of type 3 familial hemophagocytic lymphohistiocytosis. J Exp Med 204, 853-863.
Doyle, S., Vaidya, S., O'Connell, R., Dadgostar, H., Dempsey, P., Wu, T., Rao, G., Sun, R., Haberland, M., Modlin, R., et al. (2002). IRF3 mediates a TLR3/TLR4-specific antiviral gene program. Immunity 17, 251-263.
Fraser, I. D., and Germain, R. N. (2009). Navigating the network: signaling cross-talk in hematopoietic cells. Nat Immunol 10, 327-331.
Freeman, M. (2000). Feedback control of intercellular signalling in development. Nature 408, 313-319.
Heikkinen, L. S., Kazlauskas, A., Melen, K., Wagner, R., Ziegler, T., Julkunen, I., and Saksela, K. (2008). Avian and 1918 Spanish influenza a virus NS1 proteins bind to Crk/CrkL Src homology 3 domains to activate host cell signaling. J Biol Chem 283, 5719-5727.
Hennessy, E. J., Parker, A. E., and O'Neill, L. A. (2010). Targeting Toll-like receptors: emerging therapeutics? Nat Rev Drug Discov 9, 293-307.
Hrincius, E. R., Wixler, V., Wolff, T., Wagner, R., Ludwig, S., and Ehrhardt, C. (2010). CRK adaptor protein expression is required for efficient replication of avian influenza A viruses and controls JNK-mediated apoptotic responses. Cell Microbiol 12, 831-843.
Iannacone, M., Moseman, E. A., Tonti, E., Bosurgi, L., Junt, T., Henrickson, S. E., Whelan, S. P., Guidotti, L. G., and von Andrian, U. H. (2010). Subcapsular sinus macrophages prevent CNS invasion on peripheral infection with a neurotropic virus. Nature 465, 1079-1083.
Kawagoe, T., Takeuchi, O., Takabatake, Y., Kato, H., Isaka, Y., Tsujimura, T., and Akira, S. (2009). TANK is a negative regulator of Toll-like receptor signaling and is critical for the prevention of autoimmune nephritis. Nat Immunol 10, 965-972.
Keller, A. D., and Maniatis, T. (1991). Identification and characterization of a novel repressor of beta-interferon gene expression. Genes Dev 5, 868-879.
Kuan, A. P., Chamberlain, W., Malkiel, S., Lieu, H. D., Factor, S. M., Diamond, B., and Kotzin, B. L. (1999). Genetic control of autoimmune myocarditis mediated by myosin-specific antibodies. Immunogenetics 49, 79-85.
Matsui, K., Kumagai, Y., Kato, H., Sato, S., Kawagoe, T., Uematsu, S., Takeuchi, O., and Akira, S. (2006). Cutting edge: Role of TANK-binding kinase 1 and inducible IkappaB kinase in IFN responses against viruses in innate immune cells. J Immunol 177, 5785-5789.
Mross, K., Frost, A., Steinbild, S., Hedbom, S., Rentschler, J., Kaiser, R., Rouyrre, N., Trommeshauser, D., Hoesl, C. E., and Munzert, G. (2008). Phase I dose escalation and pharmacokinetic study of BI 2536, a novel Polo-like kinase 1 inhibitor, in patients with advanced solid tumors. J Clin Oncol 26, 5511-5517.
Ong, S. E., Blagoev, B., Kratchmarova, I., Kristensen, D. B., Steen, H., Pandey, A., and Mann, M. (2002). Stable isotope labeling by amino acids in cell culture, SILAC, as a simple and accurate approach to expression proteomics. Mol Cell Proteomics 1, 376-386.
Reizis, B., Bunin, A., Ghosh, H. S., Lewis, K. L., and Sisirak, V. (2011). Plasmacytoid dendritic cells: recent progress and open questions. Annu Rev Immunol 29, 163-183.
Rothlin, C. V., Ghosh, S., Zuniga, E. I., Oldstone, M. B., and Lemke, G. (2007). TAM receptors are pleiotropic inhibitors of the innate immune response. Cell 131, 1124-1136.
Santiago-Raber, M. L., Baccala, R., Haralds son, K. M., Choubey, D., Stewart, T. A., Kono, D. H., and Theofilopoulos, A. N. (2003). Type-I interferon receptor deficiency reduces lupus-like disease in NZB mice. J Exp Med 197, 777-788.
Seeburg, D. P., Pak, D., and Sheng, M. (2005). Polo-like kinases in the nervous system. Oncogene 24, 292-298.
Shalek, A. K., Robinson, J. T., Karp, E. S., Lee, J. S., Ahn, D. R., Yoon, M. H., Sutton, A., Jorgolli, M., Gertner, R. S., Gujral, T. S., et al. (2010). Vertical silicon nanowires as a universal platform for delivering biomolecules into living cells. Proc Natl Acad Sci USA 107, 1870-1875.
Sharma, K., Kumar, C., Keri, G., Breitkopf, S. B., Oppermann, F. S., and Daub, H. (2010). Quantitative analysis of kinase-proximal signaling in lipopolysaccharide-induced innate immune response. J Proteome Res 9, 2539-2549.
Strebhardt, K. (2010). Multifaceted polo-like kinases: drug targets and antitargets for cancer therapy. Nat Rev Drug Discov 9, 643-660.

Takayanagi, H., Kim, S., Matsuo, K., Suzuki, H., Suzuki, T., Sato, K., Yokochi, T., Oda, H., Nakamura, K., Ida, N., et al. (2002). RANKL maintains bone homeostasis through c-Fos-dependent induction of interferon-beta. Nature 416, 744-749.
Takeuchi, O., and Akira, S. (2010). Pattern recognition receptors and inflammation. Cell 140, 805-820.
Villen, J., and Gygi, S. P. (2008). The SCX/IMAC enrichment approach for global phosphorylation analysis by mass spectrometry. Nat Protoc 3, 1630-1638.
Vitour, D., Dabo, S., Ahmadi Pour, M., Vilasco, M., Vidalain, P. O., Jacob, Y., Mezel-Lemoine, M., Paz, S., Arguello, M., Lin, R., et al. (2009). Polo-like kinase 1 (PLK1) regulates interferon (IFN) induction by MAVS. J Biol Chem 284, 21797-21809.
Wang, L., Gordon, R. A., Huynh, L., Su, X., Park Min, K. H., Han, J., Arthur, J. S., Kalliolias, G. D., and Ivashkiv, L. B. (2010). Indirect inhibition of Toll-like receptor and type I interferon responses by ITAM-coupled receptors and integrins. Immunity 32, 518-530.
Weintz, G., Olsen, J. V., Fruhauf, K., Niedzielska, M., Amit, I., Jantsch, J., Mages, J., Frech, C., Dolken, L., Mann, M., et al. (2010). The phosphoproteome of toll-like receptor-activated macrophages. Mol Syst Biol 6, 371.
Wellcome Trust Case Control Consortium (2007). Genome-wide association study of 14,000 cases of seven common diseases and 3,000 shared controls. Nature 447, 661-678.
Xiao, N., Eidenschenk, C., Krebs, P., Brandl, K., Blasius, A. L., Xia, Y., Khovananth, K., Smart, N. G., and Beutler, B. (2009). The Tpl2 mutation Sluggish impairs type I IFN production and increases susceptibility to group B streptococcal disease. J Immunol 183, 7975-7983.
Ye, J., Chen, S., and Maniatis, T. (2011). Cardiac glycosides are potent inhibitors of interferon-beta gene expression. Nat Chem Biol 7, 25-33.
Zhang, W., Wang, J., Zhang, Y., Yuan, Y., Guan, W., Jin, C., Chen, H., Wang, X., Yang, X., and He, F. (2010). The scaffold protein TANK/1-TRAF inhibits NF-kappaB activation by recruiting polo-like kinase 1. Mol Biol Cell 21, 2500-2513.
Bengtsson, H., and Hossjer, O. (2006). Methodological study of affine transformations of gene expression data with proposed robust non-parametric multi-dimensional normalization method. BMC Bioinformatics 7, 100.
Eisen, M. B., Spellman, P. T., Brown, P. O., and Botstein, D. (1998). Cluster analysis and display of genome-wide expression patterns. Proc Natl Acad Sci USA 95, 14863-14868.
Geiss, G. K., Bumgarner, R. E., Birditt, B., Dahl, T., Dowidar, N., Dunaway, D. L., Fell, H. P., Ferree, S., George, R. D., Grogan, T., et al. (2008). Direct multiplexed measurement of gene expression with color-coded probe pairs. Nat Biotechnol 26, 317-325.
Guris, D. L., Fantes, J., Tara, D., Druker, B. J., and Imamoto, A. (2001). Mice lacking the homologue of the human 22q11.2 gene CRKL phenocopy neurocristopathies of DiGeorge syndrome. Nat Genet. 27, 293-298.
Hemmeryckx, B., Reichert, A., Watanabe, M., Kaartinen, V., de Jong, R., Pattengale, P. K., Groffen, J., and Heisterkamp, N. (2002). BCR/ABL P190 transgenic mice develop leukemia in the absence of Crkl. Oncogene 21, 3225-3231.
Hole, K., Clavijo, A., and Pineda, L. A. (2006). Detection and serotype-specific differentiation of vesicular stomatitis virus using a multiplex, real-time, reverse transcription-polymerase chain reaction assay. J Vet Diagn Invest 18, 139-146.
Inglis, K. J., Chereau, D., Brigham, E. F., Chiou, S. S., Schobel, S., Frigon, N. L., Yu, M., Caccavello, R. J., Nelson, S., Motter, R., et al. (2009). Polo-like kinase 2 (PLK2) phosphorylates alpha-synuclein at serine 129 in central nervous system. J Biol Chem 284, 2598-2602.
Lansing, T. J., McConnell, R. T., Duckett, D. R., Spehar, G. M., Knick, V. B., Hassler, D. F., Noro, N., Furuta, M., Emmitte, K. A., Gilmer, T. M., et al. (2007). In vitro biological activity of a novel small-molecule inhibitor of polo-like kinase 1. Mol Cancer Ther 6, 450-459.
Moffat, J., Grueneberg, D. A., Yang, X., Kim, S. Y., Kloepfer, A. M., Hinkle, G., Piqani, B., Eisenhaure, T. M., Luo, B., Grenier, J. K., et al. (2006). A lentiviral RNAi library for human and mouse genes applied to an arrayed viral high-content screen. Cell 124, 1283-1298.
Muller, U., Steinhoff, U., Reis, L. F., Hemmi, S., Pavlovic, J., Zinkernagel, R. M., and Aguet, M. (1994). Functional role of type I and type II interferons in antiviral defense. Science 264, 1918-1921.
Olsen, J. V., de Godoy, L. M., Li, G., Macek, B., Mortensen, P., Pesch, R., Makarov, A., Lange, 0., Horning, S., and Mann, M. (2005). Parts per million mass accuracy on an Orbitrap mass spectrometer via lock mass injection into a C-trap. Mol Cell Proteomics 4, 2010-2021.
Ong, S. E., and Mann, M. (2006). A practical recipe for stable isotope labeling by amino acids in cell culture (SILAC). Nat Protoc 1, 2650-2660.
Rappsilber, J., Mann, M., and Ishihama, Y. (2007). Protocol for micro-purification, enrichment, pre-fractionation and storage of peptides for proteomics using StageTips. Nat Protoc 2, 1896-1906.
Reindl, W., Yuan, J., Kramer, A., Strebhardt, K., and Berg, T. (2009). A pan-specific inhibitor of the polo-box domains of polo-like kinases arrests cancer cells in mitosis. Chembiochem 10, 1145-1148.
Rush, J., Moritz, A., Lee, K. A., Guo, A., Goss, V. L., Spek, E. J., Zhang, H., Zha, X. M., Polakiewicz, R. D., and Comb, M. J. (2005). Immunoaffinity profiling of tyrosine phosphorylation in cancer cells. Nat Biotechnol 23, 94-101.
Shapira, S. D., Gat-Viks, I., Shum, B. O., Dricot, A., de Grace, M. M., Wu, L., Gupta, P. B., Hao, T., Silver, S. J., Root, D. E., et al. (2009). A physical and regulatory map of host-influenza interactions reveals pathways in H1N1 infection. Cell 139, 1255-1267.
Steegmaier, M., Hoffmann, M., Baum, A., Lenart, P., Petronczki, M., Krssak, M., Gurtler, U., Garin-Chesa, P., Lieb, S., Quant, J., et al. (2007). BI 2536, a potent and selective inhibitor of polo-like kinase 1, inhibits tumor growth in vivo. Curr Biol 17, 316-322.
Storey, J. D., and Tibshirani, R. (2003). Statistical significance for genomewide studies. Proc Natl Acad Sci USA 100, 9440-9445.
Tager, A. M., Kradin, R. L., LaCamera, P., Bercury, S. D., Campanella, G. S., Leary, C. P., Polosukhin, V., Zhao, L. H., Sakamoto, H., Blackwell, T. S., et al. (2004). Inhibition of pulmonary fibrosis by the chemokine IP-10/CXCL10. Am J Respir Cell Mol Biol 31, 395-404.

1. A method of treating inflammation comprising administering to a subject in need thereof a polo-like kinase (Plk) inhibitor.

2. The method of claim 1, wherein the inflammation is associated with an innate immune response to a pathogen or pathogen derived molecule, and wherein the pathogen is a virus.

3. The method of claim 2, wherein the pathogen binds to
a) a toll-like receptor on the surface or in endomes of a dendritic cell or
b) a cytosolic RIG-1 like receptor of a dentritic cell.

4. (canceled)

5. The method of claim 1, wherein the inflammation is a symptom of a disease selected from the group consisting of viral infection, bacterial infection, autoimmune disease, or mucositis.

6. A method of decreasing anti-viral cytokine expression by a dendritic cell comprising contacting the cell with a polo-like kinase (Plk) inhibitor.

7. The method of claim 6, wherein the dendritic is in a subject in need of decreased anti-viral cytokine expression.

8. The method of claim 6, wherein the cytokine is interferon-β or CXCL-10.

9. The method of claim 1, wherein the inhibitor is specific for at least two Plks.

10. The method of claim 1, wherein the inhibitor is a pan-specific Plk inhibitor.

11. The method of claim 9, wherein the inhibitor is specific for at least Plk2 and Plk4.

12. The method of claim 10, wherein the inhibitor is BI 2536, poloxipan, or GW843682X.

13. A method of identifying genes or genetic elements associated with a pathogen specific response comprising:
a) contacting a dendritic cell with a toll-like receptor agonist; and
b) identify a gene or genetic element whose expression is modulated by step (a).

14. The method of claim 13, further comprising
c) perturbing expression of the gene or genetic element identified in step (b) in a dendritic cell that has been contacted with a toll-like receptor agonist.
d) identify a gene whose expression is modulated by step (c)

15. The method of claim 13 wherein the toll-like receptor agonist is Pam3CSK4, lipopolysaccharide, polyinosinic:polycytidylic acid, gardiquimod, or CpG.

16. The method of claim 13, wherein the pathogen is a virus, a bacteria, a fungus or a parasite.

17. The method of claim 13, wherein the pathogen specific response is an inflammatory response, and the gene or genetic element is one or more genes or genetic elements selected from the group consisting of Acpp, Batf, Ccl3, Cd70, Cebpd, Cxcl1, Cxcl2, E2f5, Il12a, Il12b, Il1a, Il1b, Il6, Inhba, Lmo4, Lztfl1, Marco, Met, Nfkb2, Nfkbiz, Ptgs2, Sh3 bp5, Sla, Slco3a1, Socs3, Stat5a, Syk, Tnf, U90926, Vnn3, Zc3h12a, and Zc3h12c

18. The method of claim 13, wherein the pathogen specific response is an antiviral response, and the gene or genetic element is one or more genes or genetic elements selected from the group consisting of 1190002H23Rik, 2900002H16Rik, Arid5a, Atm, Bbx, BC006779, Ccl4, Ccl7, Ccnd2, Cd40, Cited2, Cxcl10, Cxcl1l, Cxcl9, Dab2, Daxx, Dnmt3a, Edn1, Fgl2, Fus, Hbegf, Hdac1, Hdc, Hhex, Ifit1, Ifit2, Ifit3, Ifnb1, Iigp1, Iigp2, Il15, Il15ra, Il18, Il23a, Irf1, Irf2, Irf7, Isg15, Isg20, Lhx2, Lta, Mertk, Mx2, Nmi, Oas11, Peli1, Pla1a, Plag11, Plat, Plk2, Pm1, Rbl1, Re1, Rgs1, Rsad2, Sap30, Slfn4, Socs1, Stat1, Stat2, Tcf4, Timeless, Tlr3, Tnfsf8, Trim12, Trim21, Tsc22d1, Tyki, Usp12, and Usp25.

19. The method of claim 14, wherein the identified gene whose expression is modulated by step (c) is a signaling regulator.

20. The method of claim 20, wherein the signaling regulator is selected from the group consisting of Ikbke, Mapk9, Map3k7, Myd88, Tank, and Tbk1.

21. The method of claim 20, wherein the signaling regulator is selected from the group consisting of Crkl1, Dusp14, Map3k8, Mapkapk2, Mertk, Met, Phlpp, Plk2, Ppm1b, Ptpn1, Ptpre, Ptprj, Rgs1, Rgs2, Socs6, Sqstm1, and Syk.

22. The method of claim 14, wherein the identified gene whose expression is modulated by step (c) is a transcriptional regulator.

23. The method of claim 22, wherein the transcriptional regulator is selected from the group consisting of Adar, Aff1, Ahr, Arid1a, Arid5a, Atf3, Atf4, Bat5, Batf, Batf2, Bbx, Bcl10, Bcl3, Bhlhb2, Btg2, Cbx4, Cebpb, Cebpz, Cited2, Creb3, Daxx, Dnmt1, Dnmt3a, Dr1, E2f5, Egr1, Egr2, Elf1, Elk3, Ets2, Etv6, Fos, Foxn2, Fus, G3 bp2, Hat1, Hcls1, Hdac1, Hhex, Hif1a, Hmgn3, Hopx, Id2, Ifi35, Ifrd1, Irf1, Irf2, Irf3, Irf4, Irf5, Irf8, Irf9, Isg20, Jarid2, Jun, Klf10, Klf3, Klf4, Klf6, Lhx2, Limd1, Litaf, Lmo4, Lztfl1, Maff, Mafk, Mbnl1, Mdfic, Med21, Mtf2, Mxi1, Mybbp1a, Nab2, Nfat5, Nfe212, Nfix, Nfkb1, Nfkb2, Nfkbiz, Nmi, Nr4a1, Pa2g4, Pcaf, Plag12, Pm1, Pnrc2, Pum2, Rb1, Rbl1, Rel, Rela, Relb, Rfx5, Runx1, Sap30, Sertad1, Sfpi1, Ski1, Smyd2, Sox4, Sp1, Sp100, Stat1, Stat2, Stat4, Stat5a, Surf4, Suz12, Tcf12, Tcf4, Tcfec, Tgif1, Timeless, Tox4, Trim12, Trim21, Trim25, Trim30, Trim34, Tsc22d1, Xbp1, Zfp207, and Zfp3611.

24. The method of claim 22, wherein the transcriptional regulator is selected from the group consisting of Atf4, Bcl3, Bhlhb2, Cebpb, Cited2, Hat1, Hhex, Hmgn3, Irf1, Nfkb1, Nfkbiz, Plag12, Pnrc2, Pum2, Rela, Runx1, Ski1, Trim12, Trim21, and Trim34.

25. The method of claim 22, wherein the transcriptional regulator is selected from the group consisting of Arid1a, Atf3, Batf2, Bcl10, Btg2, E2f5, Elk3, Ets2, Etv6, Irf3, Irf4, Irf8, Irf9, Jun, Limd1, Nmi, Pml, Rbl1, Stat1, Stat2, Stat4, Timeless, and Tox4.

26. The method of claim 22, wherein the transcriptional regulator is selected from the group consisting of Adar, Aff1, Ahr, Arid5a, Bat5, Batf, Bbx, Cbx4, Cebpz, Creb3, Daxx, Dnmt1, Dnmt3a, Dr1, Egr1, Egr2, Elf1, Fos, Foxn2, Fus, G3 bp2, Hcls1, Hdac1, Hif1a, Hopx, Id2, Ifi35, Ifrd1, Irf2, Irf5, Isg20, Jarid2, Klf10, Klf3, Klf4, Klf6, Lhx2, Litaf, Lmo4, Lztfl1, Maff, Mafk, Mbnl1, Mdfic, Med21, Mtf2, Mxi1, Mybbp1a, Nab2, Nfat5, Nfe212, Nfix, Nfkb2, Nr4a1, Pa2g4, Pcaf, Rb1, Rel, Relb, Rfx5, Sap30, Sertad1, Sfpi1, Smyd2, Sox4, Sp1, Sp100, Stat5a, Surf4, Suz12, Tcf12, Tcf4, Tcfec, Tgif1, Tox4, Trim25, Trim30, Tsc22d1, Xbp1, Zfp207, and Zfp3611.

27. A method of modulating expression of one or more toll-like receptor (TLR) signature genes by perturbing expression of a control signaling molecule or a transcriptional regulator, wherein the TLR signature gene is one or more genes selected from the group consisting of 1190002H23Rik, 2900002H16Rik, Acpp, Arid5a, Atm, Batf, Bbx, BC006779, Cc13, Cc14, Cc17, Ccnd2, Cd40, Cd70, Cebpd, Cited2, Cxcl1, Cxcl10, Cxcl11, Cxcl2, Cxcl9, Dab2, Daxx, Dnmt3a, E2f5, Edn1, Fgl2, Fus, Hbegf, Hdac1, Hdc, Hhex, Ifit1, Ifit2, Ifit3, Ifnb1, Iigp1, Iigp2, Il12a, Il12b, Il15, Il15ra, Il18, Il1a, Il1b, Il23a, Il6, Inhba, Irf1, Irf2, Irf7, Isg15, Isg20, Lhx2, Lmo4, Lta, Lztfl1, Marco, Mertk, Met, Mx2, Nfkb2, Nfkbiz, Nmi, Oasl1, Peli1, Pla1a, Plag11, Plat, Plk2, Pml, Ptgs2, Rbl1, Rel, Rgs1, Rsad2, Sap30, Sh3 bp5, Sla, Slco3a1, Slfn4, Socs1, Socs3, Stat1, Stat2, Stat5a, Syk, Tcf4, Timeless, Tlr3, Tnf, Tnfsf8, Trim12, Trim21, Tsc22d1, Tyki, U90926, Usp12, Usp25, Vnn3, Zc3h12a, and Zc3h12c.

28. The method of claim 27, wherein the TLR signature gene is one or more inflammatory genes selected from the group consisting of Acpp, Batf, Cc13, Cd70, Cebpd, Cxcl1, Cxcl2, E2f5, Il12a, Il12b, Il1a, Il1b, Il6, Inhba, Lmo4, Lztfl1, Marco, Met, Nfkb2, Nfkbiz, Ptgs2, Sh3 bp5, Sla, Slco3a1, Socs3, Stat5a, Syk, Tnf, U90926, Vnn3, Zc3h12a, and Zc3h12c.

29. The method of claim 27, wherein the TLR signature gene is one or more antiviral genes selected from the group consisting of 1190002H23Rik, 2900002H16Rik, Arid5a, Atm, Bbx, BC006779, Cc14, Cc17, Ccnd2, Cd40, Cited2, Cxcl10, Cxcl1l, Cxcl9, Dab2, Daxx, Dnmt3a, Edn1, Fgl2, Fus, Hbegf, Hdac1, Hdc, Hhex, Ifit1, Ifit2, Ifit3, Ifnb1, Iigp1, Iigp2, 1115, Il15ra, Il18, Il23a, Irf1, Irf2, Irf7, Isg15, Isg20, Lhx2, Lta, Mertk, Mx2, Nmi, Oasl1, Peli1, Pla1a, Plag11, Plat, Plk2, Pml, Rbl1, Rel, Rgs1, Rsad2, Sap30, Slfn4, Socs1, Stat1, Stat2, Tcf4, Timeless, Tlr3, Tnfsf8, Trim12, Trim21, Tsc22d1, Tyki, Usp12, and Usp25.

30. The method of claim 27, wherein the signaling regulator is selected from the group consisting of Ikbke, Mapk9, Map3k7, Myd88, Tank, and Tbk1.

31. The method of claim 27, wherein the signaling regulator is selected from the group consisting of Crkl1, Dusp14, Map3k8, Mapkapk2, Mertk, Met, Phlpp, Plk2, Ppm1b, Ptpn1, Ptpre, Ptprj, Rgs1, Rgs2, Socs6, Sqstm1, and Syk.

32. The method of claim 27, wherein the signaling regulator is selected from the group consisting of Adar, Aff1, Ahr, Arid1a, Arid5a, Atf3, Atf4, Bat5, Batf, Batf2, Bbx, Bcl10, Bcl3, Bhlhb2, Btg2, Cbx4, Cebpb, Cebpz, Cited2, Creb3, Daxx, Dnmt1, Dnmt3a, Dr1, E2f5, Egr1, Egr2, Elf1, Elk3, Ets2, Etv6, Fos, Foxn2, Fus, G3 bp2, Hat1, Hcls1, Hdac1, Hhex, Hif1a, Hmgn3, Hopx, Id2, Ifi35, Ifrd1, Irf1, Irf2, Irf3, Irf4, Irf5, Irf8, Irf9, Isg20, Jarid2, Jun, Klf10, Klf3, Klf4, Klf6, Lhx2, Limd1, Litaf, Lmo4, Lztfl1, Maff, Mafk, Mbnl1, Mdfic, Med21, Mtf2, Mxi1, Mybbp1a, Nab2, Nfat5, Nfe212, Nfix, Nfkb1, Nfkb2, Nfkbiz, Nmi, Nr4a1, Pa2g4, Pcaf, Plag12, Pml, Pnrc2, Pum2, Rb, Rbl1, Rel, Rela, Relb, Rfx5, Runx1, Sap30, Sertad1, Sfpi1, Ski1, Smyd2, Sox4, Sp1, Sp100, Stat1, Stat2, Stat4, Stat5a, Surf4, Suz12, Tcf12, Tcf4, Tcfec, Tgif1, Timeless, Tox4, Trim12, Trim21, Trim25, Trim30, Trim34, Tsc22d1, Xbp1, Zfp207, and Zfp3611.

33. The method of claim 27, wherein the signaling regulator is selected from the group consisting of Atf4, Bcl3, Bhlhb2, Cebpb, Cited2, Hat1, Hhex, Hmgn3, Irf1, Nfkb1, Nfkbiz, Plag12, Pnrc2, Pum2, Rela, Runx1, Ski1, Trim12, Trim21, and Trim34.

34. The method of claim 27, wherein the transcriptional regulator is selected from the group consisting of Arid1a, Atf3, Batf2, Bcl10, Btg2, E2f5, Elk3, Ets2, Etv6, Irf3, Irf4, Irf8, Irf9, Jun, Limd1, Nmi, Pml, Rbl1, Stat1, Stat2, Stat4, Timeless, and Tox4.

35. The method of claim 27, wherein the transcriptional regulator is selected from the group consisting of Adar, Aff1, Ahr, Arid5a, Bat5, Batf, Bbx, Cbx4, Cebpz, Creb3, Daxx, Dnmt1, Dnmt3a, Dr1, Egr1, Egr2, Elf1, Fos, Foxn2, Fus, G3 bp2, Hcls1, Hdac1, Hif1a, Hopx, Id2, Ifi35, Ifrd1, Irf2, Irf5, Isg20, Jarid2, Klf10, Klf3, Klf4, Klf6, Lhx2, Litaf, Lmo4, Lztfl1, Maff, Mafk, Mbnl1, Mdfic, Med21, Mtf2, Mxi1, Mybbp1a, Nab2, Nfat5, Nfe212, Nfix, Nfkb2, Nr4a1, Pa2g4, Pcaf, Rb1, Rel, Relb, Rfx5, Sap30, Sertad1, Sfpi1, Smyd2, Sox4, Sp1, Sp100, Stat5a, Surf4, Suz12, Tcf12, Tcf4, Tcfec, Tgif1, Tox4, Trim25, Trim30, Tsc22d1, Xbp1, Zfp207, and Zfp3611.


SEQUENCE LISTING (2 sequences)

SEQ ID NO: 1 - DNA, 25 bp, Artificial Sequence
  Chemically synthesized oligonucleotide
tgatacagtacaattattttgggac

SEQ ID NO: 2 - DNA, 24 bp, Artificial Sequence
  Chemically synthesized oligonucleotide
gagactttctgttacgggatctgg